(19) United States
(12) Patent Application Publication
(10) Pub. No.: US 2024/0180457 A1
(43) Pub. Date: Jun. 6, 2024

(54) BIOLOGICAL INFORMATION MEASUREMENT DEVICE, BIOLOGICAL INFORMATION MEASUREMENT METHOD, AND BIOLOGICAL INFORMATION MEASUREMENT SYSTEM

(71) Applicant: SEIKO EPSON CORPORATION, TOKYO (JP)

(72) Inventors: Ayae SAWADO, Kai-shi (JP); Hiroki KOBAYASHI, HARA-MURA (JP)

(21) Appl. No.: 18/530,562

(22) Filed: Dec. 6, 2023

(30) Foreign Application Priority Data
Dec. 6, 2022 (JP) 2022-194683

(51) Int. Cl. A61B 5/1455; A61B 5/00

(52) U.S. Cl. CPC A61B 5/14552; A61B 5/4818; A61B 5/7246; A61B 2562/0242

(57) ABSTRACT

A biological information measurement device includes: a light emitting unit including a first light emitting element configured to emit red light, a second light emitting element configured to emit infrared light, and a third light emitting element configured to emit green light; a light receiving unit configured to receive the red light, the infrared light, and the green light emitted by the light emitting unit, and to generate a first light receiving signal based on the red light, a second light receiving signal based on the infrared light, and a third light receiving signal based on the green light; and a controller configured to measure a pulse wave. The controller operates the light emitting unit and the light receiving unit in a first mode of measuring the pulse wave using the third light receiving signal or in a second mode of measuring the pulse wave using the first light receiving signal or the second light receiving signal.

The present application is based on, and claims priority from JP Application Serial Number 2022-194683, filed Dec. 6, 2022, the disclosure of which is hereby incorporated by reference herein in its entirety.

### BACKGROUND

### 1. Technical Field

The present disclosure relates to a biological information measurement device, a biological information measurement method, and a biological information measurement system.

### 2. Related Art

A measurement device that non-invasively measures biological information of a subject is known. The measurement device disclosed in JP-A-2022-86227 measures a pulse wave and an oxygen saturation concentration. The measurement device includes a first light emitter, a second light emitter, and a third light emitter. The first light emitter emits green light having a green wavelength band to a measurement site. The second light emitter emits red light having a red wavelength band to the measurement site. The third light emitter emits near infrared light having a near infrared wavelength band to the measurement site. The measurement device specifies the pulse wave based on a detection signal representing a received light intensity of the green light. The measurement device specifies the oxygen saturation concentration by analyzing a detection signal representing a received light intensity of the red light and a detection signal representing a received light intensity of the near infrared light. The measurement device specifies the oxygen saturation concentration using a pulsation component of an artery.

In a biological information measurement device using the first light emitter, the second light emitter, and the third light emitter, power consumption may increase.

### SUMMARY

A biological information measurement device according to the present disclosure includes: a light emitting unit including a first light emitting element configured to emit red light, a second light emitting element configured to emit infrared light, and a third light emitting element configured to emit green light; a light receiving unit configured to receive the red light, the infrared light, and the green light emitted by the light emitting unit, and to generate a first light receiving signal based on the red light, a second light receiving signal based on the infrared light, and a third light receiving signal based on the green light; and a controller configured to measure a pulse wave and an oxygen saturation concentration based on the first light receiving signal, the second light receiving signal, and the third light receiving signal. The controller operates the light emitting unit and the light receiving unit in a first mode of measuring the pulse wave using the third light receiving signal or in a second mode of measuring the pulse wave using the first light receiving signal or the second light receiving signal.

A biological information measurement method according to the present disclosure includes: causing a first light emitting element configured to emit red light and a second light emitting element configured to emit infrared light to emit light; generating a first light receiving signal based on the red light and a second light receiving signal based on the infrared light; operating in a second mode of measuring a pulse wave using the first light receiving signal or the second light receiving signal; and operating by switching to a first mode of measuring the pulse wave using a third light receiving signal based on green light emitted by a third light emitting element when a switching signal is received.

A biological information measurement system according to the present disclosure includes a biological information measurement device and a control device. The biological information measurement device includes a light emitting unit including a first light emitting element configured to emit red light, a second light emitting element configured to emit infrared light, and a third light emitting element configured to emit green light, a light receiving unit configured to receive the red light, the infrared light, and the green light emitted by the light emitting unit, and to generate a first light receiving signal based on the red light, a second light receiving signal based on the infrared light, and a third light receiving signal based on the green light, a controller configured to operate the light emitting unit and the light receiving unit, and a communication unit configured to transmit the first light receiving signal, the second light receiving signal, and the third light receiving signal. The control device includes a terminal communication circuit configured to receive the first light receiving signal, the second light receiving signal, and the third light receiving signal, and a data controller configured to calculate a pulse wave using any one of the first light receiving signal, the second light receiving signal, and the third light receiving signal, and to evaluate an irregular pulse. The controller operates the light emitting unit and the light receiving unit in a first mode of generating the third light receiving signal or in a second mode of generating the first light receiving signal and the second light receiving signal.

### BRIEF DESCRIPTION OF THE DRAWINGS

FIG. 1 is a diagram showing a schematic configuration of a measurement system.

FIG. 2 is a diagram showing a schematic configuration of a measurement surface of a measurement device.

FIG. 3 is a diagram showing a block configuration of the measurement device.

FIG. 4 is a diagram schematically showing a detection signal.

FIG. 5 is a diagram showing a relationship between a frequency and a signal intensity of each detection signal in a predetermined time.

FIG. 6 is a diagram showing red light detection signal data and infrared light detection signal data.

FIG. 7 is a diagram showing red light detection signal data and infrared light detection signal data.

FIG. 8 is a flowchart for calculating a pulse wave.

FIG. 9 is a diagram showing a block configuration of the measurement device.

FIG. 10 is a flowchart for measuring a pulse wave.

FIG. 11 is a flowchart for measuring a pulse wave.

FIG. 12 is a diagram showing a block configuration of the measurement system.

FIG. 13 is a diagram showing an operation of the measurement device in the measurement system.

FIG. 14 is a diagram showing an operation of a tablet terminal in the measurement system.

### DESCRIPTION OF EMBODIMENTS

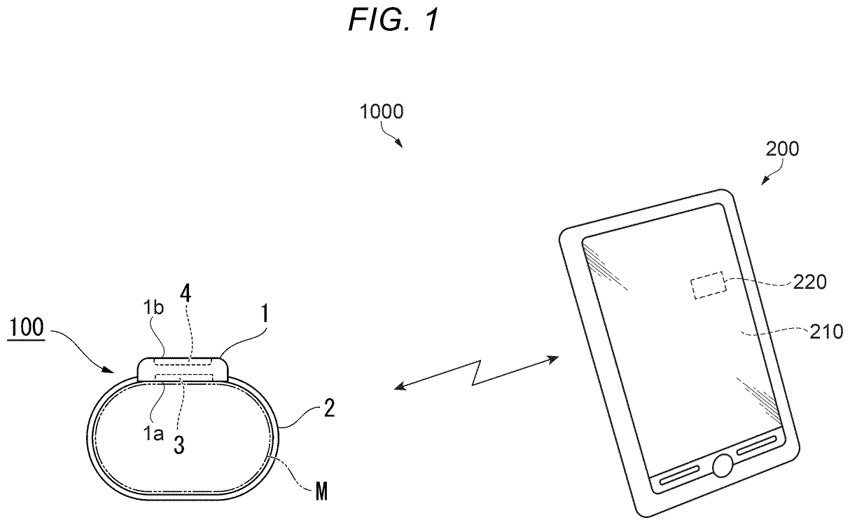

FIG. 1 shows a schematic configuration of a measurement system 1000. The measurement system 1000 evaluates an irregular pulse. The measurement system 1000 evaluates the irregular pulse using a pulse wave as an index. The irregular pulse is evaluated using a beat interval or the like. When an irregular pulse is evaluated using the measurement system 1000, pulse waves of at least 7 days or more are measured. The measurement system 1000 may evaluate a sleep apnea syndrome. The sleep apnea syndrome is referred to as SAS. The measurement system 1000 evaluates the sleep apnea syndrome using a blood oxygen saturation concentration (SpO₂) as an index. The blood oxygen saturation concentration is hereinafter referred to as an oxygen saturation concentration. When the measurement system 1000 is used to evaluate the sleep apnea syndrome, the oxygen saturation concentration is measured over one to three nights. The sleep apnea syndrome is evaluated based on a low-concentration period indicating an oxygen saturation concentration lower than a predetermined value or the number of times of low-concentration periods. The measurement system 1000 corresponds to an example of a biological information measurement system.

The measurement system 1000 includes a measurement device 100 and a tablet terminal 200. The measurement device 100 and the tablet terminal 200 are communicably connected. The measurement system 1000 shown in FIG. 1 performs wireless communication connection between the measurement device 100 and the tablet terminal 200. The connection between the measurement device 100 and the tablet terminal 200 is not limited to wireless. The measurement device 100 and the tablet terminal 200 may be communicably connected by wire. The measurement device 100 corresponds to an example of a biological information measurement device. The tablet terminal 200 corresponds to an example of a control device.

The measurement device 100 measures biological information of a user M such as a human or various types of data related to the biological information. The measurement device 100 is a watch type portable device worn on a measurement site of the user M. The measurement device 100 shown in FIG. 1 is worn on, for example, a wrist of the user M. The measurement device 100 measures biological information such as a pulse wave including a pulse interval and an oxygen saturation concentration over time. A pulse wave interval is expressed as post pacing interval (PPI). The pulse wave indicates a change over time in a volume of a blood vessel in conjunction with a heart beat. The oxygen saturation concentration indicates a proportion of hemoglobin bound to oxygen in hemoglobin in arterial blood of the user M. The oxygen saturation concentration is an index for evaluating a breathing function of the user M. The measurement device 100 may measure the biological information other than the pulse wave and the oxygen saturation concentration. The measurement device 100 measures, for example, a glucose concentration in the arterial blood and an alcohol concentration in the arterial blood. The measurement device 100 includes a housing 1 and a belt 2. The housing 1 accommodates a detection unit 3 and a display panel 4.

The housing 1 is an exterior housing accommodating a unit or the like in the measurement device 100. The housing 1 has a measurement surface 1a and a display surface 1b. The measurement surface 1a is a surface facing the measurement site of the user M. The measurement surface 1a comes into contact with the measurement site of the user M. The display surface 1b is a surface visible to the user M. In addition to the detection unit 3 and the display panel 4, the housing 1 accommodates a control unit 30, a memory 40, and the like, which will be described later.

The belt 2 is a member that is used when the housing 1 is worn on the measurement site of the user M. The belt 2 is attached to, for example, a side surface of the housing 1. The belt 2 is wound around the measurement site, so that the housing 1 is worn at the measurement site of the user M. The measurement device 100 shown in FIG. 1 includes the belt 2, but is not limited thereto. The measurement device 100 may not include the belt 2. The measurement device 100 may be worn on a chest, an arm, or the like of the user M with a tape or the like.

The detection unit 3 is disposed at the measurement surface 1a of the housing 1. The detection unit 3 is disposed at a position facing the measurement site of the user M. The detection unit 3 acquires various types of data used when measuring the biological information.

The display panel 4 is disposed at the display surface 1b of the housing 1. The display panel 4 is visible to the user M. The display panel 4 displays various types of the measured biological information. The display panel 4 may display information other than the biological information, such as a reliability index and time of the biological information. The display panel 4 corresponds to an example of a display unit.

The tablet terminal 200 evaluates an irregular pulse. The tablet terminal 200 evaluates the sleep apnea syndrome. The tablet terminal 200 acquires various types of data transmitted from the measurement device 100. The tablet terminal 200 evaluates the irregular pulse using various types of data. The tablet terminal 200 evaluates the sleep apnea syndrome using various types of data. The measurement system 1000 shown in FIG. 1 includes the tablet terminal 200, but is not limited thereto. The measurement system 1000 may include a personal computer, a smartphone, a dedicated terminal for analyzing biological information, or the like, instead of the tablet terminal 200. The tablet terminal 200 includes a display 210 and a terminal control unit 220.

The display 210 displays an evaluation result of the irregular pulse. The display 210 may display an evaluation result of the sleep apnea syndrome. The display 210 may display biological information such an as oxygen saturation concentration and a pulse interval in a chart.

The terminal control unit 220 evaluates the irregular pulse. The terminal control unit 220 evaluates the sleep apnea syndrome. The terminal control unit 220 acquires various types of data transmitted from the measurement device 100. The terminal control unit 220 evaluates an irregular pulse using various types of data. The terminal control unit 220 evaluates the sleep apnea syndrome using various types of data. The terminal control unit 220 generates chart data related to biological information such as the pulse wave and the oxygen saturation concentration. The terminal control unit 220 controls the display 210 to display an evaluation result of various types of biological information and the biological information in a chart on the display 210. The terminal control unit 220 corresponds to an example of a data controller.

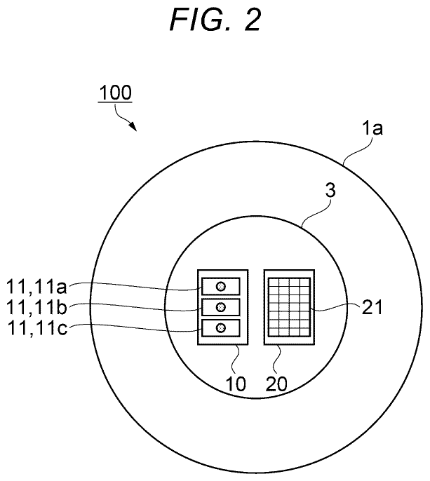

FIG. 2 shows a schematic configuration of the measurement surface 1a of the measurement device 100. FIG. 2 shows a schematic configuration of the measurement surface 1a of the measurement device 100 when viewed from an outside. The measurement surface 1a shown in FIG. 2 is formed in a circular shape, but is not limited thereto. The measurement surface 1a may be formed in various shapes such as a square shape and an elliptical shape. The detection unit 3 is disposed at the measurement surface 1a. The detection unit 3 includes a light emitting element unit 10 and a light receiving element unit 20. The light emitting element unit 10 corresponds to an example of a light emitting unit. The light receiving element unit 20 corresponds to an example of a light receiving unit.

The light emitting element unit 10 emits light toward the measurement site of the user M. The light emitting element unit 10 shown in FIG. 2 includes three light emitting elements 11. The three light emitting elements 11 are a red light emitting element 11a, an infrared light emitting element 11b, and a green light emitting element 11c. The red light emitting element 11a, the infrared light emitting element 11b, and the green light emitting element 11c emit light having different wavelength ranges. An arrangement of the three light emitting elements 11 is appropriately set. The number of light emitting elements 11 is not limited to three. Four or more light emitting elements 11 may be provided at the light emitting element unit 10.

The light emitting element 11 is implemented with a bare chip type or shell type light emitting diode (LED). The light emitting element 11 may be implemented with a laser diode. A configuration of the light emitting element 11 is appropriately set according to a wavelength range of emitted light.

The red light emitting element 11a emits red light RL toward the measurement site of the user M. The red light emitting element 11a emits the red light RL in a wavelength range of 600 nm to 800 nm toward the measurement site. The red light RL is, for example, light having a peak wavelength of 660 nm. The red light emitting element 11a corresponds to an example of a first light emitting element.

The infrared light emitting element 11b emits infrared light NL toward the measurement site of the user M. The infrared light emitting element 11b emits the infrared light NL in a wavelength range of 800 nm to 1300 nm toward the measurement site. The infrared light NL is, for example, near infrared light having a peak wavelength of 905 nm. The infrared light emitting element 11b corresponds to an example of a second light emitting element.

The green light emitting element 11c emits green light GL toward the measurement site of the user M. The green light emitting element 11c emits the green light GL in a wavelength range of 520 nm to 550 nm toward the measurement site. The green light GL is, for example, light having a peak wavelength of 520 nm. The green light emitting element 11c corresponds to an example of a third light emitting element.

The light receiving element unit 20 receives various types of light emitted by the light emitting element unit 10. The light receiving element unit 20 includes a light receiving element 21 that receives various types of light. The light receiving element 21 receives transmitted light or reflected light of light emitted by the light emitting element unit 10. The transmitted light is light transmitted through the user M. The reflected light is light reflected inside the user M and transmitted through an inside of the user M. The light receiving element 21 includes one or a plurality of photodiodes.

### First Embodiment

A first embodiment shows a first measurement device 100a as an example of the measurement device 100. The first embodiment shows a biological information measurement method using the first measurement device 100a. In the first embodiment, the pulse wave and the oxygen saturation concentration are measured using the first measurement device 100a. The first measurement device 100a transmits the pulse wave and the oxygen saturation concentration to the tablet terminal 200. The tablet terminal 200 receives the pulse wave and the oxygen saturation concentration. The tablet terminal 200 evaluates the irregular pulse using the pulse wave. The tablet terminal 200 evaluates the sleep apnea syndrome using the oxygen saturation concentration.

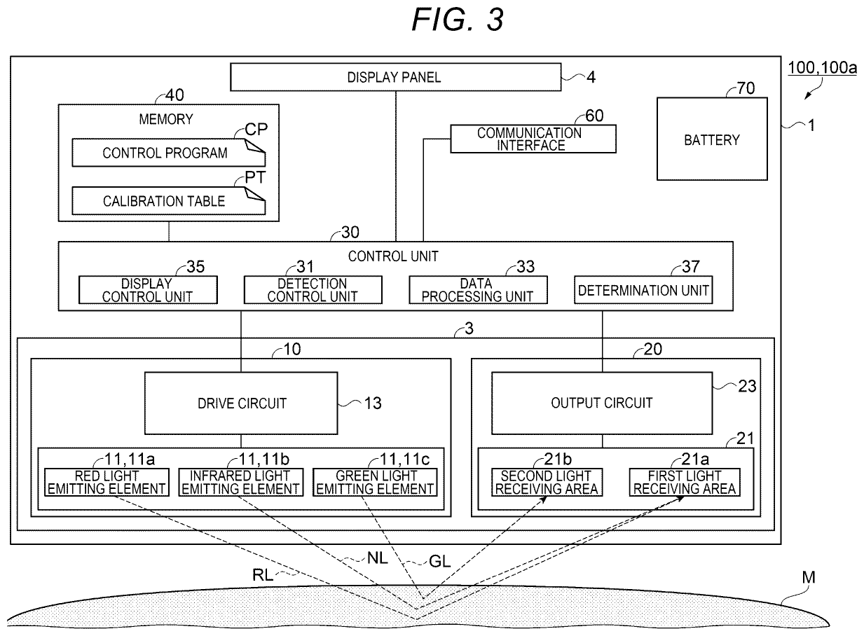

FIG. 3 shows a block configuration of the measurement device 100. FIG. 3 shows a block configuration of the first measurement device 100a as an example of the measurement device 100. FIG. 3 shows the first measurement device 100a excluding the belt 2. The first measurement device 100a accommodates various units or the like in the housing 1. The first measurement device 100a includes the detection unit 3, the control unit 30, the memory 40, the display panel 4, a communication interface 60, and a battery 70.

The detection unit 3 is an optical sensor module that detects, as a detection signal, data related to biological information measured using light in various wavelength ranges. The detection unit 3 includes the light emitting element unit 10 and the light receiving element unit 20.

The light emitting element unit 10 includes the red light emitting element 11a, the infrared light emitting element 11b, the green light emitting element 11c, and a drive circuit 13. The red light emitting element 11a, the infrared light emitting element 11b, and the green light emitting element 11c are disposed at positions facing the measurement site of the user M. The light emitting element unit 10 operates under control of the control unit 30.

The drive circuit 13 drives the plurality of light emitting elements 11. The drive circuit 13 causes the plurality of light emitting elements 11 to emit light under the control of the control unit 30. The drive circuit 13 shown in FIG. 3 causes the red light emitting element 11a, the infrared light emitting element 11b, and the green light emitting element 11c to emit light.

The light receiving element unit 20 includes the light receiving element 21 and an output circuit 23. The light receiving element unit 20 operates under control of the control unit 30. The light receiving element 21 receives light emitted by the light emitting element 11 and reflected by the measurement site of the user M. The light receiving element 21 receives the red light RL, the infrared light NL, and the green light GL reflected by the measurement site of the user M. The light receiving element 21 is divided into a plurality of regions. The light receiving element 21 may be divided into the plurality of regions using an optical filter (not shown). The light receiving element 21 shown in FIG. 3 is divided into a first light receiving area 21a and a second light receiving area 21b.

The first light receiving area 21a receives the red light RL and the infrared light NL. The first light receiving area 21a receives the red light RL emitted by the red light emitting element 11a and reflected by the measurement site of the user M. The first light receiving area 21a receives the infrared light NL emitted by the infrared light emitting element 11b and reflected by the measurement site of the user M. The first light receiving area 21a may receive at least one piece of the red light RL and the infrared light NL via the optical filter. The first light receiving area 21a may alternately receive the red light RL and the infrared light NL in a time-division manner.

The second light receiving area 21b receives the green light GL. The second light receiving area 21b receives the green light GL emitted by the green light emitting element 11c and reflected by the measurement site of the user M. The second light receiving area 21b may receive the green light GL via the optical filter.

The light receiving element 21 shown in FIG. 3 receives the red light RL and the infrared light NL in the first light receiving area 21a, but is not limited thereto. A third light receiving area different from the first light receiving area 21a and the second light receiving area 21b may be formed. The red light RL or the infrared light NL may be received by the third light receiving area. At this time, when the third light receiving area receives the infrared light NL, the first light receiving area 21a receives the red light RL. The light receiving element 21 may not be divided into the plurality of regions. The light receiving element 21 may receive the red light RL, the infrared light NL, and the green light GL in a time-division manner.

The output circuit 23 outputs, to the control unit 30, a detection signal based on the light received by the light receiving element 21. The output circuit 23 generates a detection signal by performing processing such as analog-to-digital conversion on received light intensity data of the light received by the light receiving element 21. The output circuit 23 generates a red light detection signal based on the red light RL received by the first light receiving area 21a. The output circuit 23 generates an infrared light detection signal based on the infrared light NL received by the first light receiving area 21a. The output circuit 23 generates a green light detection signal based on the green light GL received by the second light receiving area 21b. The red light detection signal corresponds to an example of a first light receiving signal. The infrared light detection signal corresponds to an example of a second light receiving signal. The green light detection signal corresponds to an example of a third light receiving signal.

The control unit 30 is a controller that controls operations of various units. The control unit 30 is, for example, a processor including a central processing unit (CPU). The control unit 30 may include one or a plurality of processors. The control unit 30 may include a semi-conductor memory such as a random access memory (RAM) or a read only memory (ROM). The semi-conductor memory functions as a work area for the control unit 30. The control unit 30 functions as a detection control unit 31, a data processing unit 33, a display control unit 35, and a determination unit 37 by executing a control program CP stored in the memory 40. The detection control unit 31, the data processing unit 33, the display control unit 35, and the determination unit 37 are functional units. The control unit 30 operates the light emitting element unit 10 and the light receiving element unit 20 under control of each functional unit. The control unit 30 corresponds to an example of a controller.

The detection control unit 31 controls the light emitting element unit 10 and the light receiving element unit 20. The detection control unit 31 operates the light emitting element unit 10 and the light receiving element unit 20. The detection control unit 31 adjusts a light emitting timing, a light off timing, a light amount, or the like of the light emitting element 11 via the drive circuit 13. The detection control unit 31 controls a light receiving timing, a light receiving time, digital-to-analog conversion, or the like of various types of light for the light receiving element unit 20. The detection control unit 31 controls the light emitting element unit 10 and the light receiving element unit 20 to cause the detection unit 3 to generate a red light detection signal, an infrared light detection signal, and a green light detection signal. The detection control unit 31 controls the light emitting element unit 10 and the light receiving element unit 20 based on a determination result of the determination unit 37. The control of the detection control unit 31 based on the determination result of the determination unit 37 will be described later.

The detection control unit 31 operates the light emitting element unit 10 and the light receiving element unit 20 in the first measurement mode or in the second measurement mode. The first measurement mode is an operation mode different from the second measurement mode.

The first measurement mode is a mode of measuring a pulse wave using the green light detection signal. The detection control unit 31 causes the light emitting element unit 10 to cause the green light emitting element 11c to emit light. The detection control unit 31 causes the green light emitting element 11c to emit light via the drive circuit 13. The light receiving element unit 20 receives the green light GL emitted from the green light emitting element 11c. The second light receiving area 21b receives the green light GL. The output circuit 23 generates a green light detection signal based on the green light GL. The output circuit 23 outputs the green light detection signal to the control unit 30. The data processing unit 33 of the control unit 30 measures the pulse wave using the green light detection signal. The first measurement mode corresponds to an example of a first mode.

The detection control unit 31 may not generate the red light detection signal or the infrared light detection signal when the light emitting element unit 10 and the light receiving element unit 20 are operated in the first measurement mode. The detection control unit 31 does not generate the red light detection signal or the infrared light detection signal by turning off the red light emitting element 11a and the infrared light emitting element 11b. The detection control unit 31 may cause the light receiving element unit 20 not to receive the red light RL or the infrared light NL. The detection control unit 31 may cause the output circuit 23 to erase the red light detection signal and the infrared light detection signal without outputting the red light detection signal or the infrared light detection signal to the control unit 30. The detection control unit 31 preferably turns off the red light emitting element 11a and the infrared light emitting element 11b. Power consumption is reduced by turned off the red light emitting element 11a and the infrared light emitting element 11b.

The second measurement mode is a mode of measuring the pulse wave using the red light detection signal or the infrared light detection signal. The detection control unit 31 causes the light emitting element unit 10 to cause the red light emitting element 11a and the infrared light emitting element 11b to emit light. The detection control unit 31 causes the red light emitting element 11a and the infrared light emitting element 11b to emit light via the drive circuit 13. The light receiving element unit 20 receives red light RL emitted from the red light emitting element 11a. The light receiving element unit 20 receives the infrared light NL emitted from the infrared light emitting element 11b. The first light receiving area 21a receives the red light RL and the infrared light NL. The output circuit 23 generates a red light detection signal based on the red light RL. The output circuit 23 generates an infrared light detection signal based on the infrared light NL. The output circuit 23 outputs the red light detection signal and the infrared light detection signal to the control unit 30. The data processing unit 33 of the control unit 30 measures the pulse wave using the red light detection signal or the infrared light detection signal. The data processing unit 33 compares, for example, a signal intensity of the red light detection signal with a signal intensity of the infrared light detection signal, and selects the red light detection signal or the infrared light detection signal. The data processing unit 33 measures the pulse wave using a selected detection signal. The data processing unit 33 measures the oxygen saturation concentration using the red light detection signal and the infrared light detection signal. The second measurement mode corresponds to an example of a second mode.

The detection control unit 31 may not generate the green light detection signal when the light emitting element unit 10 and the light receiving element unit 20 are operated in the second measurement mode. The detection control unit 31 does not generate the green light detection signal by turning off the green light emitting element 11c. The detection control unit 31 may cause the light receiving element unit 20 to execute an operation of not receiving the green light GL. The detection control unit 31 may cause the output circuit 23 to erase the green light detection signal without transmitting the green light detection signal to the control unit 30. The detection control unit 31 preferably turns off the green light emitting element 11c. Power consumption is reduced by turning off the green light emitting element 11c.

The data processing unit 33 processes the detection signal output from the light receiving element unit 20. The data processing unit 33 acquires the red light detection signal, the infrared light detection signal, and the green light detection signal from the light receiving element unit 20.

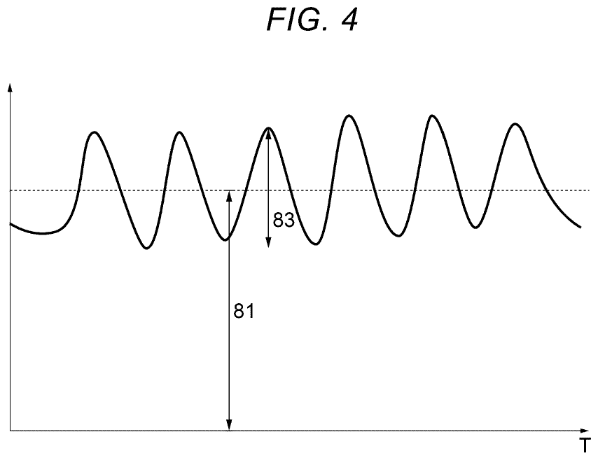

The data processing unit 33 acquires a DC component and an AC component from the detection signal. FIG. 4 schematically shows a detection signal. A horizontal axis in FIG. 4 indicates time. A vertical axis in FIG. 4 indicates a signal intensity of the detection signal. FIG. 4 schematically shows an example of the detection signal output from the output circuit 23.

The detection signal is data of signal intensity detected at predetermined measurement time intervals. The signal intensity is detected n times per second. n is an integer of 1 or more. n is 16 as an example. The signal intensity includes DC component data 81 as a DC component and AC component data 83 as an AC component. The data processing unit 33 separates the DC component data 81 and the AC component data 83 based on the signal intensity. The data processing unit 33 calculates the AC component data 83 by performing time-frequency analysis.

The data processing unit 33 performs time-frequency analysis such as short time Fourier transform on the detection signal. The data processing unit 33 analyzes frequency information by performing the short time Fourier transform on the detection signal. The data processing unit 33 obtains a spectrogram in a predetermined frequency range by performing the short time Fourier transform on the detection signal. The predetermined frequency range is a range including a frequency of a pulse wave. The predetermined frequency range is, for example, a range of 0.5 Hz to 2 Hz. The predetermined frequency range is appropriately adjusted according to a wavelength range of light subjected to the short time Fourier transform. The data processing unit 33 performs the short time Fourier transform on the red light detection signal to obtain a red light spectrogram. The data processing unit 33 performs the short time Fourier transform on the infrared light detection signal to obtain an infrared light spectrogram. The data processing unit 33 performs the short time Fourier transform on the green light detection signal to obtain a green light spectrogram. The data processing unit 33 corresponds to an example of a controller.

The time-frequency analysis executed by the data processing unit 33 is not limited to the short time Fourier transform. The method is not limited as long as the frequency information for the detection signal can be analyzed. The data processing unit 33 may perform, for example, wavelet transform.

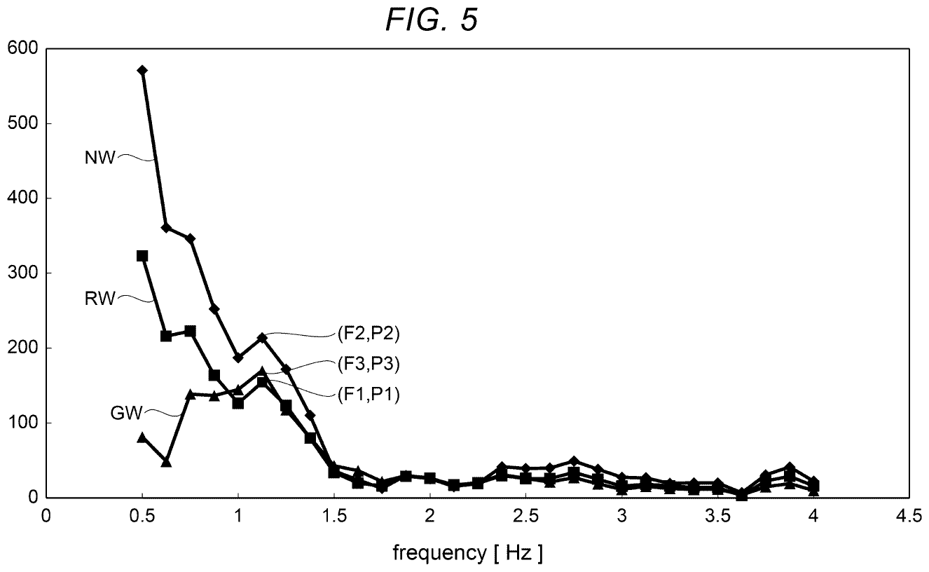

FIG. 5 shows a relationship between a frequency and a signal intensity of each detection signal in a predetermined time. FIG. 5 shows a part of a result of the short time Fourier transform. FIG. 5 shows red light data RW, infrared light data NW, and green light data GW in the predetermined time. The red light data RW indicates a relationship between a frequency and a signal intensity of the red light RL at the predetermined time.

The infrared light data NW indicates a relationship between a frequency and a signal intensity of the infrared light NL at the predetermined time. The green light data GW indicates a relationship between a frequency and a signal intensity of the green light GL at the predetermined time.

The red light data RW indicates a first peak value P1 at a first frequency F1. The first frequency F1 corresponds to a frequency of a pulse wave. The data processing unit 33 acquires a signal intensity at each time at the first frequency F1 as a red light detection signal intensity.

The infrared light data NW indicates a second peak value P2 at a second frequency F2. The second frequency F2 is the same as or approximate to the first frequency F1. The second frequency F2 corresponds to a frequency of a pulse wave. The data processing unit 33 acquires a signal intensity at each time at the second frequency F2 as an infrared light detection signal intensity.

The green light data GW indicates a third peak value P3 at a third frequency F3. The third frequency F3 is the same as or approximate to the first frequency F1 or the second frequency F2. The third frequency F3 corresponds to a frequency of a pulse wave. The data processing unit 33 acquires a signal intensity at each time at the third frequency F3 as a green light detection signal intensity.

The data processing unit 33 measures the pulse wave using the green light detection signal. The data processing unit 33 measures the pulse wave using the green light detection signal intensity at the third frequency F3. The data processing unit 33 measures the pulse wave using the red light detection signal or the infrared light detection signal. The data processing unit 33 measures the pulse wave using the red light detection signal intensity at the first frequency F1. The data processing unit 33 measures the pulse wave using the infrared light detection signal intensity at the second frequency F2.

The data processing unit 33 acquires the red light detection signal intensity and the infrared light detection signal intensity at each time. The data processing unit 33 measures a fluctuation component amplitude ratio using the red light detection signal intensity and the infrared light detection signal intensity. The fluctuation component amplitude ratio is a ratio between a red light transmitted amount and an infrared light transmitted amount. The red light transmitted amount is a light amount of the red light RL, which is emitted from the red light emitting element 11a, is transmitted through the measurement site of the user M, and reaches the light receiving element 21. The infrared light transmitted amount is a light amount of the infrared light NL, which is emitted from the infrared light emitting element 11b, is transmitted through the measurement site of the user M, and reaches the light receiving element 21. The fluctuation component amplitude ratio is calculated by the following formula (1).

R=(AC_{Red}/DC_{Red})/(AC_{IR}/DC_{IR}) (1)

Here, R indicates the fluctuation component amplitude ratio. AC_{Red} indicates an intensity of an AC component of the red light detection signal. DC_{Red} indicates an intensity of a DC component of the red light detection signal. AC_{IR} indicates an intensity of an AC component of the infrared light detection signal. DC_{IR} indicates an intensity of a DC component of the infrared light detection signal.

The intensity of the AC component of the red light detection signal is the red light detection signal intensity. The intensity of the DC component of the red light detection signal is a DC component at a predetermined time separated from the red light detection signal. The intensity of the AC component of the infrared light detection signal is the infrared light detection signal intensity. The intensity of the DC component of the infrared light detection signal is a DC component at a predetermined time separated from the infrared light detection signal.

The data processing unit 33 calculates the oxygen saturation concentration based on the calculated fluctuation component amplitude ratio. The data processing unit 33 refers to a calibration table PT stored in the memory 40 to obtain a value of the oxygen saturation concentration corresponding to the fluctuation component amplitude ratio. The data processing unit 33 determines, as the oxygen saturation concentration, a value of the oxygen saturation concentration corresponding to the fluctuation component amplitude ratio. The data processing unit 33 measures the oxygen saturation concentration using the red light detection signal and the infrared light detection signal.

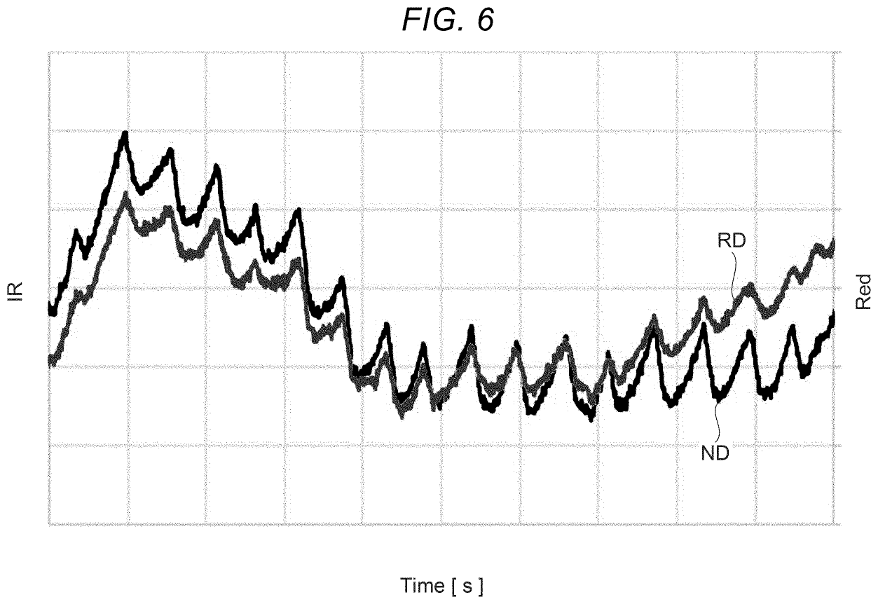

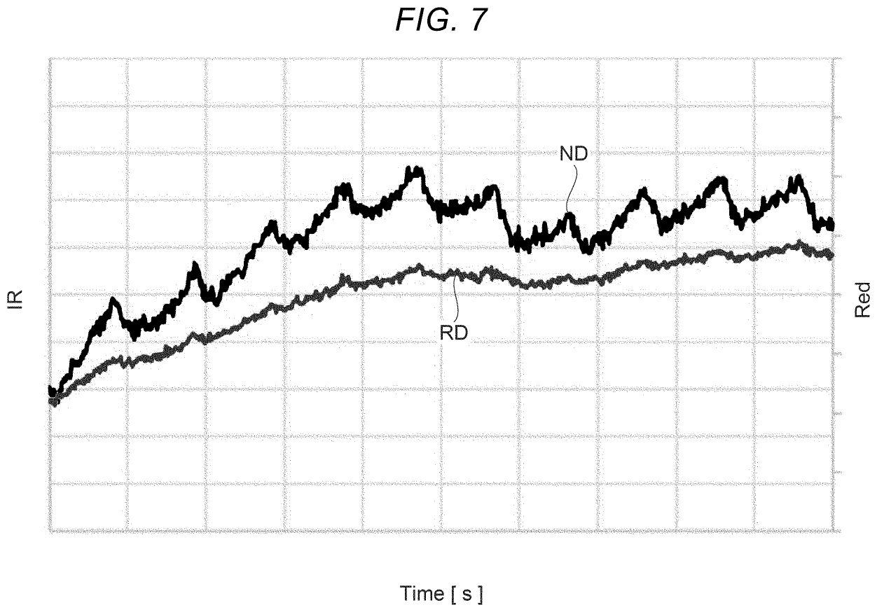

FIGS. 6 and 7 show red light detection signal data RD and infrared light detection signal data ND. FIG. 6 shows the red light detection signal data RD and the infrared light detection signal data ND when the user M is in a sleeping state. FIG. 7 shows the red light detection signal data RD and the infrared light detection signal data ND when the user M is not in a sleeping state. The red light detection signal data RD indicates a change in the red light detection signal over time. The infrared light detection signal data ND indicates a change in the infrared light detection signal over time. Left vertical axes in FIGS. 6 and 7 indicate signal values of the infrared light detection signal. Right vertical axes in FIGS. 6 and 7 indicate signal values of the red light detection signal.

As shown in FIG. 6, when the user M is in a sleeping state, sensitivities of an AC component of the red light detection signal data RD and of an AC component of the infrared light detection signal data ND are improved. A correlation between the AC component of the red light detection signal data RD and the AC component of the infrared light detection signal data ND is improved. The data processing unit 33 can accurately measure a pulse wave using the AC component of the red light detection signal data RD or the AC component of the infrared light detection signal data ND.

On the other hand, as shown in FIG. 7, when the user M is not in the sleeping state, the sensitivity of the AC component of the red light detection signal data RD and the sensitivity of the AC component of the infrared light detection signal data ND decrease. The sensitivity of the AC component of the red light detection signal data RD and the sensitivity of the AC component of the infrared light detection signal data ND decrease due to an influence of noise generated by body motion or the like. The sensitivity of the AC component of the red light detection signal data RD is, for example, a ratio of the signal intensity of the AC component to a signal intensity of a DC component in the red light detection signal. The sensitivity of the AC component of the infrared light detection signal data ND is, for example, a ratio of the signal intensity of the AC component to a signal intensity of a DC component in the infrared light detection signal. It is difficult for the data processing unit 33 to accurately measure a pulse wave using the AC component of the red light detection signal data RD or the AC component of the infrared light detection signal data ND.

As shown in FIG. 7, when the user M is not in the sleeping state, the correlation between the AC component of the red light detection signal data RD and the AC component of the infrared light detection : signal data ND decreases. The correlation between the AC component of the red light detection signal data RD and the AC component of the infrared light detection signal data ND decreases due to an influence of noise generated by body motion or the like. It is difficult for the data processing unit 33 to accurately measure a pulse wave using the AC component of the red light detection signal data RD or the AC component of the infrared light detection signal data ND.

The data processing unit 33 may calculate the sensitivity of the AC component of the red light detection signal data RD and the sensitivity of the AC component of the infrared light detection signal data ND as sensitivity data. The sensitivity data is calculated using the red light detection signal and the infrared light detection signal. The data processing unit 33 may calculate the correlation between the AC component of the red light detection signal data RD and the AC component of the infrared light detection signal data ND as a correlation coefficient. The correlation coefficient is calculated using the red light detection signal and the infrared light detection signal. The sensitivity data and the correlation coefficient calculated by the data processing unit 33 are used by the determination unit 37. The sensitivity data and the correlation coefficient are collectively referred to as quality data. The quality data corresponds to an example of signal quality data.

As an example, the data processing unit 33 calculates a correlation coefficient using the red light detection signal and the infrared light detection signal. The data processing unit 33 calculates a correlation coefficient between red light detection signal data RD and infrared light detection signal data ND. The detection control unit 31 uses a correlation coefficient as an index for determining whether to operate the light emitting element unit 10 and the light receiving element unit 20 in the first measurement mode or in the second measurement mode. The correlation coefficient can be used as an index for determining whether the user M is in a sleeping state. When the user M is in an active state, the correlation coefficient decreases due to an influence of noise generated by body motion or the like. When the user M is in a sleeping state, the influence of noise caused by body motion or the like is reduced, and the correlation coefficient is improved. The data processing unit 33 calculates the correlation coefficient using a correlation coefficient calculation formula represented by the following formula (2). The correlation coefficient corresponds to an example of correlation data.

$$r = \frac{{\sum }_{n = 1}^{N} ( {x}_{n} - {x}_{ave} ) ( {y}_{n} - {y}_{ave} )}{\sqrt{{\sum }_{n = 1}^{N} {( {x}_{n} - {x}_{ave} )}^{2} {\sum }_{n = 1}^{N} {( {y}_{n} - {y}_{ave} )}^{2}}} \qquad ( 2 )$$

Here, r represents the correlation coefficient. N represents the number of red light detection signals used for calculating the correlation coefficient. xₙ indicates a red light detection signal at each measurement time. n is an integer of 1 or more. xₐᵥₑ indicates an average value of red light detection signals within a predetermined time. yₙ represents an infrared light detection signal at each measurement time. yₐᵥₑ indicates an average value of infrared light detection signals within a predetermined time. The predetermined time is, for example, 8 seconds. When the red light detection signal and the infrared light detection signal are measured k times per second, N is 8×k. k is an integer of 1 or more.

The data processing unit 33 outputs the pulse wave and the oxygen saturation concentration to the display control unit 35. The data processing unit 33 outputs the pulse wave and the oxygen saturation concentration to the tablet terminal 200 via the communication interface 60.

The display control unit 35 controls display of the display panel 4. The display control unit 35 displays various images on the display panel 4 by transmitting display data to the display panel 4. The display control unit 35 acquires the pulse wave and the oxygen saturation concentration from the data processing unit 33 at a predetermined timing. The display control unit 35 generates display data including the pulse wave. The display control unit 35 may generate display data including the oxygen saturation concentration. The display control unit 35 outputs the display data including the pulse wave, the oxygen saturation concentration, and the like to the display panel 4. The display control unit 35 displays the pulse wave and the oxygen saturation concentration based on the display data on the display panel 4. The display control unit 35 corresponds to an example of a controller.

The determination unit 37 determines whether the user M is in a sleeping state. The determination unit 37 acquires sensitivity data or a correlation function from the data processing unit 33. The determination unit 37 determines whether the user M is in the sleeping state based on the sensitivity data or the correlation function.

The determination unit 37 acquires sensitivity data as an example. The determination unit 37 acquires the sensitivity of the AC component of the red light detection signal data RD or the sensitivity of the AC component of the infrared light detection signal data ND as the sensitivity data. The determination unit 37 determines whether the user M is in a sleeping state by comparing a preset sensitivity data threshold value with the sensitivity data. When the sensitivity data is smaller than the sensitivity data threshold value, the determination unit 37 determines that the user M is not in the sleeping state. When the sensitivity data is equal to or larger than the sensitivity data threshold value, the determination unit 37 determines that the user M is in the sleeping state. The determination unit 37 corresponds to an example of the controller. The sensitivity data threshold value corresponds to an example of a threshold value.

The determination unit 37 acquires the correlation coefficient as an example. The determination unit 37 determines whether the user M is in a sleeping state by comparing a preset correlation coefficient threshold value with the correlation coefficient. When the correlation coefficient is smaller than the correlation coefficient threshold value, the determination unit 37 determines that the user M is not in the sleeping state. The determination unit 37 determines that the user M is in the active state. When the correlation coefficient is equal to or larger than the correlation coefficient threshold value, the determination unit 37 determines that the user M is in the sleeping state. The correlation coefficient threshold value is an example of the threshold value. The correlation coefficient threshold value corresponds to an example of a correlation data threshold value. The sensitivity data threshold value and the correlation coefficient threshold value are collectively referred to as a quality data threshold value.

The determination unit 37 generates a determination signal such as a sleeping state signal indicating that the user M is in the sleeping state based on a determination result. The determination unit 37 may generate a sleep displacement signal indicating displacement from an active state to a sleeping state as the determination signal based on a determination result. The determination unit 37 transmits the sleeping state signal or the sleep displacement signal to either the detection control unit 31 or the data processing unit 33. The determination unit 37 may transmit the sleeping state signal or the sleep displacement signal to the detection control unit 31 and the data processing unit 33. The determination unit 37 may generate an active state signal indicating that the user M is in an active state as the determination signal. The determination unit 37 may generate an activity displacement signal indicating displacement from a sleeping state to an active state as the determination signal. The determination unit 37 transmits the active state signal or the activity displacement signal to either the detection control unit 31 or the data processing unit 33. The determination unit 37 may transmit the active state signal or the activity displacement signal to the detection control unit 31 and the data processing unit 33.

The detection control unit 31 operates the light emitting element unit 10 and the light receiving element unit 20 based on the sleeping state signal. When the sleeping state signal is received, the detection control unit 31 operates the light emitting element unit 10 and the light receiving element unit 20 in the second measurement mode. When the sleeping state signal is not received, the detection control unit 31 operates the light emitting element unit 10 and the light receiving element unit 20 in the first measurement mode. When the sleeping state signal and the active state signal are received, the detection control unit 31 controls driving of the light emitting element 11 based on the sleeping state signal and the active state signal. When the active state signal is received, the detection control unit 31 causes the green light emitting element 11c to emit light. When the active state signal is received, the detection control unit 31 turns off the red light emitting element 11a and the infrared light emitting element 11b. When the sleeping state signal is received, the detection control unit 31 causes the red light emitting element 11a and the infrared light emitting element 11b to emit light. When the sleeping state signal is received, the detection control unit 31 turns off the green light emitting element 11c.

The detection control unit 31 may operate the light emitting element unit 10 and the light receiving element unit 20 based on the sleep displacement signal and the activity displacement signal. When the sleep displacement signal is received, the detection control unit 31 operates the light emitting element unit 10 and the light receiving element unit 20 in the second measurement mode. As an example, the detection control unit 31 causes the red light emitting element 11a and the infrared light emitting element 11b to emit light. The detection control unit 31 turns off the green light emitting element 11c. When the activity displacement signal is received, the detection control unit 31 operates the light emitting element unit 10 and the light receiving element unit 20 in the first measurement mode. As an example, the detection control unit 31 causes the green light emitting element 11c to emit light. The detection control unit 31 turns off the red light emitting element 11a and the infrared light emitting element 11b. When the sleep displacement signal is received while operating the light emitting element unit 10 and the light receiving element unit 20 in the first measurement mode, the detection control unit 31 changes the first measurement mode to the second measurement mode and operates the light emitting element unit 10 and the light receiving element unit 20.

The detection control unit 31 switches a mode of operating the light emitting element unit 10 and the light receiving element unit 20 depending on whether the sensitivity data is equal to or larger than the sensitivity data threshold value. The detection control unit 31 switches the mode of operating the light emitting element unit 10 and the light receiving element unit 20 depending on whether the correlation coefficient is equal to or larger than the correlation coefficient threshold value.

When the determination unit 37 determines that the correlation coefficient equal to or larger than the correlation coefficient threshold value, the detection control unit 31 causes the red light emitting element 11a and the infrared light emitting element 11b to emit light. The detection control unit 31 may cause the green light emitting element 11c to emit light or turn off the green light emitting element 11c. The detection control unit 31 preferably turns off the green light emitting element 11c. An increase in power consumption due to light emission of the green light emitting element 11c is prevented. The light receiving element 21 receives the red light RL and the infrared light NL. The data processing unit 33 receives the red light detection signal based on the red light RL. The data processing unit 33 receives the infrared light detection signal based on the infrared light NL. When the light receiving element 21 receives the green light GL, the detection control unit 31 does not cause the output circuit 23 to generate a green light detection signal. Alternatively, the detection control unit 31 does not cause the output circuit 23 to transmit the green light detection signal to the data processing unit 33. The data processing unit 33 measures the pulse wave using the red light detection signal or the infrared light detection signal. The data processing unit 33 measures the oxygen saturation concentration using the red light detection signal and the infrared light detection signal. Since the data processing unit 33 does not execute data processing using the green light detection signal, an increase in power consumption is prevented.

When the determination unit 37 determines that the correlation coefficient is smaller than the correlation coefficient threshold value, the detection control unit 31 causes the green light emitting element 11c to emit light. The detection control unit 31 may cause the red light emitting element 11a and the infrared light emitting element 11b to emit light or turn off the red light emitting element 11a and the infrared light emitting element 11b. The detection control unit 31 preferably turns off the red light emitting element 11a and the infrared light emitting element 11b. An increase in power consumption due to light emission of the red light emitting element 11a and the infrared light emitting element 11b is prevented. The light receiving element 21 receives the green light GL. The data processing unit 33 receives a green light detection signal based on the green light GL. When the light receiving element 21 receives the red light RL and the infrared light NL, the detection control unit 31 does not cause the output circuit 23 to generate a red light detection signal or an infrared light detection signal. Alternatively, the detection control unit 31 does not cause the output circuit 23 to transmit the red light detection signal or the infrared light detection signal to the data processing unit 33. The data processing unit 33 measures the pulse wave using the green light detection signal. The data processing unit 33 does not measure the oxygen saturation concentration. Since the data processing unit 33 does not execute data processing using the red light detection signal or the infrared light detection signal, an increase in power consumption is prevented.

The memory 40 stores various types of data. The memory 40 stores control data for operating various units, various types of data measured by the control unit 30, or the like. The memory 40 may store various types of evaluation data used in the determination unit 37 and the like. The memory 40 may store the pulse wave, the oxygen saturation concentration, and the like measured by the data processing unit 33. The memory 40 stores a sensitivity data threshold value or a correlation coefficient threshold value as evaluation data. The memory 40 stores the control program CP that operates in the control unit 30. The memory 40 may store the correlation coefficient calculation formula. The memory 40 stores the calibration table PT referred to by the data processing unit 33. The memory 40 may store a conversion formula or a conversion table. The memory 40 is implemented by a ROM, a RAM, or the like. The memory 40 corresponds to an example of a storage unit.

The control program CP is executed by the control unit 30 to operate various functional units. The control program CP causes the control unit 30 to operate as the detection control unit 31, the data processing unit 33, the display control unit 35, and the determination unit 37. The control program CP may cause the control unit 30 to operate as a functional unit other than the detection control unit 31, the data processing unit 33, the display control unit 35, and the determination unit 37.

The calibration table PT is a table that stores the fluctuation component amplitude ratio and the oxygen saturation concentration in association with each other. The calibration table PT shows a relationship between the fluctuation component amplitude ratio and the oxygen saturation concentration. The calibration table PT is created in advance by a manufacturer of the measurement device 100. The data processing unit 33 refers to the calibration table PT to determine the oxygen saturation concentration corresponding to the calculated fluctuation component amplitude ratio. The calibration table PT corresponds to an example of a calibration curve table.

The memory 40 may store a calibration formula instead of the calibration table PT. The calibration formula is a relational expression between the fluctuation component amplitude ratio and the oxygen saturation concentration. The data processing unit 33 measures the oxygen saturation concentration corresponding to the calculated fluctuation component amplitude ratio using the calibration formula.

The display panel 4 displays various images. The display panel 4 displays the pulse wave and data related to the pulse wave under control of the display control unit 35. The data related to the pulse wave is, for example, a pulse rate and a beat interval. The data related to the pulse wave is generated by the data processing unit 33. The display panel 4 may display the oxygen saturation concentration under the control of the display control unit 35. The display panel 4 displays the oxygen saturation concentration based on display data output from the display control unit 35. The display panel 4 includes a liquid crystal display, an organic electro- luminescence (EL) display, or the like.

The communication interface 60 is an interface circuit for performing communication connection with the tablet terminal 200. The communication interface 60 is connected to the tablet terminal 200 in a wired or wireless manner according to a predetermined protocol. The communication interface 60 includes, for example, a connection port for wired communication or an antenna for wireless communication. The communication interface 60 receives control data, information related to the user M, and the like from the tablet terminal 200. The communication interface 60 transmits various types of biological data such as the pulse wave and the oxygen saturation concentration to the tablet terminal 200. The communication interface 60 transmits various types of measurement data such as a red light detection signal and an infrared light detection signal. The communication interface 60 may be communicably connected to an external device other than the tablet terminal 200. The communication interface 60 corresponds to an example of a communication unit.

The battery 70 supplies power to various units and the like of the first measurement device 100a. The battery 70 is implemented by a lithium primary battery, a lithium ion secondary battery, or the like. The battery 70 is preferably a rechargeable lithium ion secondary battery. The lithium ion secondary battery is charged by wire or wirelessly. When the battery 70 is used as a power supply source of the first measurement device 100a, the control unit 30 preferably operates the light emitting element unit 10 and the light receiving element unit 20 in the first measurement mode or in the second measurement mode. The control unit 30 can reduce power consumption by the light emitting element unit 10 and the light receiving element unit 20.

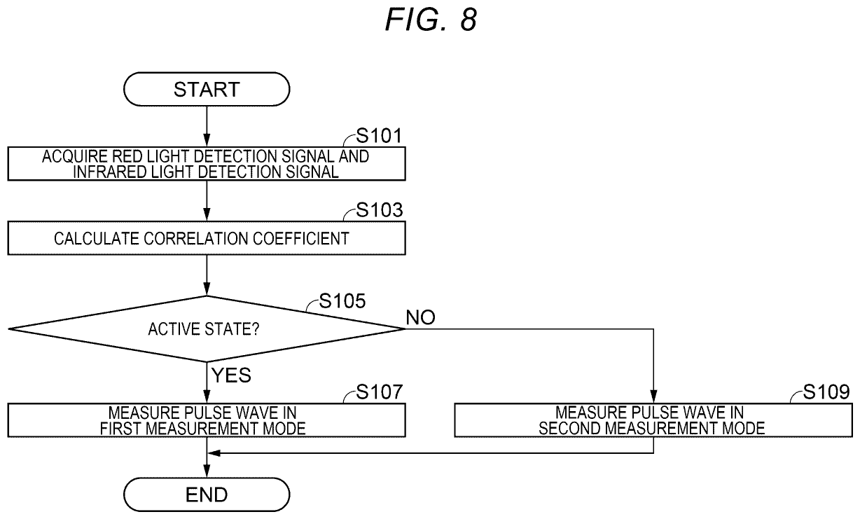

FIG. 8 shows a flowchart for measuring a pulse wave. The flowchart shown in FIG. 8 is an example of the biological information measurement method. FIG. 8 shows the biological information measurement method executed by the first measurement device 100a. The biological information measurement method shown in FIG. 8 is appropriately executed at predetermined time intervals.

In step S101, the control unit 30 acquires a red light detection signal and an infrared light detection signal. The detection control unit 31 of the control unit 30 causes the red light emitting element 11a and the infrared light emitting element 11b to emit light at predetermined time intervals. The red light emitting element 11a emits the red light RL to the measurement site of the user M. The infrared light emitting element 11b emits the infrared light NL to the measurement site of the user M. The light receiving element 21 of the light receiving element unit 20 receives the red light RL and the infrared light NL. The output circuit 23 generates a red light detection signal based on the red light RL and an infrared light detection signal based on the infrared light NL. The output circuit 23 outputs the red light detection signal and the infrared light detection signal to the data processing unit 33. The data processing unit 33 of the control unit 30 acquires the red light detection signal and the infrared light detection signal. When the red light detection signal and the infrared light detection signal are acquired, the control unit 30 may or may not cause the green light emitting element 11c to emit light.

When the red light detection signal and the infrared light detection signal are acquired, the control unit 30 calculates a correlation coefficient in step S103. The data processing unit 33 calculates the correlation coefficient using the red light detection signal and the infrared light detection signal. In FIG. 8, the control unit 30 calculates the correlation coefficient, and may calculate sensitivity data instead of the correlation coefficient. The data processing unit 33 transmits the correlation coefficient to the determination unit 37. The determination unit 37 acquires the correlation coefficient.

After calculating the correlation coefficient, the control unit 30 determines whether the user M is in an active state in step S105. The control unit 30 determines whether the correlation coefficient is equal to or larger than the correlation coefficient threshold value. The control unit 30 determines whether the user M is in the active state using the correlation coefficient. The determination unit 37 reads the correlation coefficient threshold value stored in the memory 40. The determination unit 37 compares the correlation coefficient with the correlation coefficient threshold value. When the correlation coefficient is smaller than the correlation coefficient threshold value, the determination unit 37 determines that the user M is in the active state. The determination unit 37 transmits an active state signal to the detection control unit 31. The determination unit 37 may transmit the activity displacement signal to the detection control unit 31. The active state signal and the activity displacement signal correspond to an example of a switching signal. The control unit 30 proceeds to step S107 (step S105: YES). When the correlation coefficient is equal to or larger than the correlation coefficient threshold value, the determination unit 37 determines that the user M is in a sleeping state. The determination unit 37 determines that the user M is not in the active state. The determination unit 37 transmits a sleeping state signal to the detection control unit 31. The determination unit 37 may transmit a sleep displacement signal to the detection control unit 31. The sleeping state signal and the sleep displacement signal correspond to an example of the switching signal. The control unit 30 proceeds to step S109 (step S105: NO). In step S105 in FIG. 8, the correlation coefficient and the correlation coefficient threshold value are used to determine whether the user M is in the active state, but the present disclosure is not limited thereto. The sensitivity data and the sensitivity data threshold value may be used for determination.

In step S107, the control unit 30 measures the pulse wave in the first measurement mode. The detection control unit 31 of the control unit 30 operates the light emitting element unit 10 and the light receiving element unit 20 in the first measurement mode. The detection control unit 31 causes the green light emitting element 11c to emit light via the drive circuit 13. The green light emitting element 11c emits the green light GL to the measurement site of the user M. The light receiving element 21 of the light receiving element unit 20 receives the green light GL. The output circuit 23 generates a green light detection signal based on the green light GL. The output circuit 23 transmits the green light detection signal to the data processing unit 33. The data processing unit 33 acquires the green light detection signal. The data processing unit 33 measures the pulse wave using the green light detection signal.

When the pulse wave is measured in the first measurement mode, the control unit 30 may cause the red light emitting element 11a and the infrared light emitting element 11b to emit light or turn off the red light emitting element 11a and the infrared light emitting element 11b. The control unit 30 causes the red light emitting element 11a and the infrared light emitting element 11b to emit light at predetermined time intervals. In the first measurement mode, a light emission period in which the red light emitting element 11a and the infrared light emitting element 11b emit light is preferably shorter than a light-off period in which the red light emitting element 11a and the infrared light emitting element 11b are turned off. Power consumption of the first measurement device 100a is reduced. The control unit 30 acquires the red light detection signal and the infrared light detection signal in the light emission period.

In step S109, the control unit 30 measures the pulse wave in the second measurement mode. The detection control unit 31 of the control unit 30 operates the light emitting element unit 10 and the light receiving element unit 20 in the second measurement mode. The detection control unit 31 causes the red light emitting element 11a and the infrared light emitting element 11b to emit light via the drive circuit 13. The red light emitting element 11a emits the red light RL to the measurement site of the user M. The infrared light emitting element 11b emits the infrared light NL to the measurement site of the user M. The light receiving element 21 of the light receiving element unit 20 receives the red light RL and the infrared light NL. The output circuit 23 generates a red light detection signal based on the red light RL. The output circuit 23 generates an infrared light detection signal based on the infrared light NL. The output circuit 23 outputs the red light detection signal and the infrared light detection signal to the data processing unit 33. The data processing unit 33 acquires the red light detection signal and the infrared light detection signal. The data processing unit 33 measures the pulse wave using the red light detection signal or the infrared light detection signal. The data processing unit 33 measures the oxygen saturation concentration using the red light detection signal and the infrared light detection signal.

When the pulse wave is measured in the second measurement mode, the control unit 30 may cause the green light emitting element 11c to emit light or turn off the green light emitting element 11c. The control unit 30 preferably turns off the green light emitting element 11c. The power consumption of the first measurement device 100a is reduced by turning off the green light emitting element 11c. When the green light emitting element 11c emits light, the output circuit 23 preferably does not generate a green light detection signal. The data processing unit 33 does not execute data processing of measuring the pulse wave using the green light detection signal. The first measurement device 100a can prevent an increase in power consumption due to data processing.

The first measurement device 100a includes: the light emitting element unit 10 including the red light emitting element 11a configured to emit the red light RL, the infrared light emitting element 11b configured to emit the infrared light NL, and the green light emitting element 11c configured to emit the green light GL; the light receiving receiving element unit 20 configured to receive the red light RL, the infrared light NL, and the green light GL emitted by the light emitting element unit 10, and to generate a red light detection signal based on the red light RL, an infrared light detection signal based on the infrared light NL, and a green light detection signal based on the green light GL; and the control unit 30 configured to measure a pulse wave and an oxygen saturation concentration based on the red light detection signal, the infrared light detection signal, and the green light detection signal. The control unit 30 operates the light emitting element unit 10 and the light receiving element unit 20 in the first measurement mode of measuring the pulse wave using the green light detection signal or in the second measurement mode of measuring the pulse wave using the red light detection signal or the infrared light detection signal.

When the first measurement device 100a operates in the second measurement mode, it is possible to omit data processing of measuring the pulse wave using the green light detection signal. Since the data processing of measuring the pulse wave using the green light detection signal is omitted, the first measurement device 100a can reduce the power consumption related to the data processing.

The control unit 30 calculates quality data using the red light detection signal and the infrared light detection signal, operates the light emitting element unit 10 and the light receiving element unit 20 in the second measurement mode when the quality data is equal to or larger than a predetermined quality data threshold value, and operates the light emitting element unit 10 and the light receiving element unit 20 in the first measurement mode when the quality data is smaller than the quality data threshold value.

The first measurement device 100a measures the pulse wave using the red light detection signal or the infrared light detection signal when the user M is in the sleeping state. The red light detection signal or the infrared light detection signal is more likely to be affected by noise caused by body motion or the like than the green light detection signal. The first measurement device 100a can measure the pulse wave using the red light detection signal or the infrared light detection signal when the red light detection signal or the infrared light detection signal are less likely to be affected by noise. The first measurement device 100a can reduce a decrease in pulse wave measurement accuracy.

The quality data is preferably a correlation coefficient between the red light detection signal and the infrared light detection signal, and the quality data threshold value is preferably a correlation coefficient threshold value.

The first measurement device 100a can accurately determine whether the user M is in the sleeping state using the correlation coefficient.

The control unit 30 preferably turns off the red light emitting element 11a and the infrared light emitting element 11b when operating the light emitting element unit 10 and the light receiving element unit 20 in the first measurement mode.

The first measurement device 100a can reduce power consumption when the light emitting element unit 10 and the light receiving element unit 20 are operated in the first measurement mode.

The control unit 30 preferably turns off the green light emitting element 11c when operating the light emitting element unit 10 and the light receiving element unit 20 in the second measurement mode.

The first measurement device 100a can reduce power consumption when the light emitting element unit 10 and the light receiving element unit 20 are operated in the second measurement mode.

The biological information measurement method includes: causing the red light emitting element 11a configured to emit the red light RL and the infrared light emitting element 11b configured to emit the infrared light NL to emit light; generating the red light detection signal based on the red light RL and the infrared light detection signal based on the infrared light NL; operating in the second measurement mode of measuring the pulse wave using the red light detection signal or the infrared light detection signal; and operating by switching to the first measurement mode of measuring the pulse wave using the green light detection signal based on the green light GL emitted by the green light emitting element 11c when the active state signal or the activity displacement signal is received.

The first measurement device 100a switches the detection signal according to whether the user M is in a sleeping state, and measures the pulse wave. The first measurement device 100a can measure the pulse wave while preventing a decrease in measurement accuracy according to a state of the user M.

When the biological information measurement method operates in the second measurement mode, the oxygen saturation concentration is measured using the red light detection signal and the infrared light detection signal.

The first measurement device 100a can measure the oxygen saturation concentration used to evaluate the sleep apnea syndrome .

### Second Embodiment

A second embodiment shows a second measurement device 100b as an example of the measurement device 100. The second embodiment shows the biological information measurement method using the second measurement device 100b. In the second embodiment, the pulse wave and the oxygen saturation concentration are measured using the second measurement device 100b. The second measurement device 100b transmits the pulse wave and the oxygen saturation concentration to the tablet terminal 200. The tablet terminal 200 receives the pulse wave and the oxygen saturation concentration. The tablet terminal 200 evaluates the irregular pulse using the pulse wave. The tablet terminal 200 evaluates the sleep apnea syndrome using the oxygen saturation concentration.

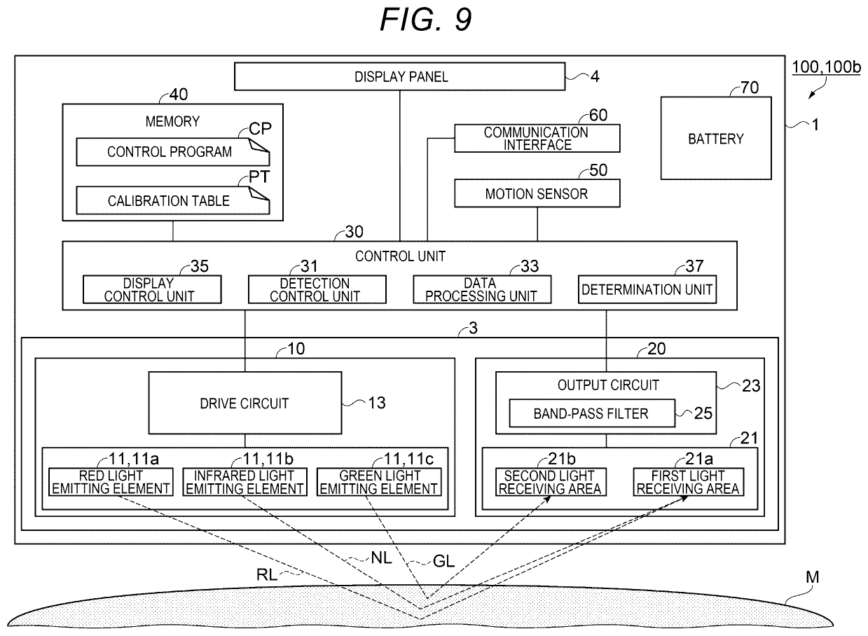

FIG. 9 shows a block configuration of the measurement device 100. FIG. 9 shows a block configuration of the second measurement device 100b as an example of the measurement device 100. FIG. 9 shows the second measurement device 100b excluding the belt 2. The second measurement device 100b accommodates various units or the like in the housing 1. The second measurement device 100b includes the detection unit 3, the control unit 30, the memory 40, the display panel 4, a motion sensor 50, the communication interface 60, and the battery 70. A configuration of the second measurement device 100b is the same as a configuration of the first measurement device 100a except for the output circuit 23 of the light receiving element unit 20 and the motion sensor 50.

The output circuit 23 includes a band-pass filter 25. The band-pass filter 25 extracts an AC component from the received light intensity data. The band-pass filter 25 separates the AC component and a DC component by extracting the AC component from the received light intensity data. The AC component corresponds to the AC component data 83 shown in FIG. 4. The DC component corresponds to the DC component data 81 shown in FIG. 4. The band-pass filter 25 outputs the separated AC component and DC component as detection signals to the control unit 30.

The band-pass filter 25 extracts a red light AC component from the red light RL received by the first light receiving area 21a. The band-pass filter 25 separates the red light AC component and a red light DC component by extracting the red light AC component. The band-pass filter 25 extracts an infrared light AC component from the infrared light NL received by the first light receiving area 21a. The band-pass filter 25 separates the infrared light AC component and an infrared light DC component by extracting the infrared light AC component. The output circuit 23 outputs the red light AC component and the red light DC component as red light detection signals to the control unit 30. The output circuit 23 outputs the infrared light AC component and the infrared light DC component as infrared light detection signals to the control unit 30.

The band-pass filter 25 extracts the green light AC component from the green light GL received by the second light receiving area 21b. The band-pass filter 25 separates the green light AC component and a green light DC component by extracting the green light AC component. The output circuit 23 outputs the green light AC component and the green light DC component as green light detection signals to the control unit 30.

The motion sensor 50 measures body motion of the user M. The motion sensor 50 generates motion data indicating the body motion of the user M. The motion sensor 50 outputs the generated motion data as a measurement result to the control unit 30. A configuration of the motion sensor 50 is not limited as long as the motion sensor 50 detects the body motion of the user M. As the motion sensor 50, an acceleration sensor, a direction sensor, a gyro sensor, a position sensor such as a positioning satellite signal receiver, or the like is used. The motion sensor 50 is preferably an acceleration sensor. The acceleration sensor detects movement of a part on which the second measurement device 100b is worn. When the part of the user M moves, the acceleration sensor detects movement of the user M. The motion sensor 50 corresponds to an example of a body motion sensor.

The control unit 30 receives the motion data. The determination unit 37 of the control unit 30 acquires the motion data. The determination unit 37 determines whether the user M is in a sleeping state based on the motion data. As an example, the determination unit 37 acquires an output data amount, an accumulated data amount, and the like per unit time from the motion sensor 50 as the motion data. The determination unit 37 determines whether the user M is in a sleeping state by comparing a preset body motion amount threshold value with the motion data. When the motion data is smaller than the body motion amount threshold value, the determination unit 37 determines that the user M is in the sleeping state. When the motion data is equal to or larger than the body motion amount threshold value, the determination unit 37 determines that the user M is not in the sleeping state. The determination unit 37 determines that the user M is in the active state.

The determination unit 37 generates a determination signal such as a sleeping state signal indicating that the user M is in the sleeping state based on the motion data. The determination unit 37 may generate a sleep displacement signal indicating displacement from an active state to a sleeping state as the determination signal based on the motion data. The determination unit 37 transmits the sleeping state signal or the sleep displacement signal to either the detection control unit 31 or the data processing unit 33. The determination unit 37 may transmit the sleeping state signal or the sleep displacement signal to the detection control unit 31 and the data processing unit 33. The determination unit 37 may generate an active state signal indicating that the user M is in an active state as a determination signal. The determination unit 37 may generate an activity displacement signal indicating displacement from a sleeping state to an active state as the determination signal. The determination unit 37 transmits the active state signal or the activity displacement signal to either the detection control unit 31 or the data processing unit 33. The determination unit 37 may transmit the active state signal or the activity displacement signal to the detection control unit 31 and the data processing unit 33.

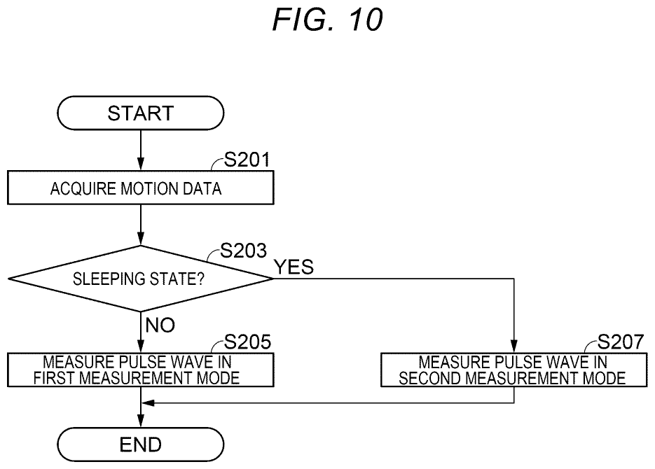

FIG. 10 shows a flowchart for measuring a pulse wave. The flowchart shown in FIG. 10 is an example of the biological information measurement method. FIG. 10 shows the biological information measurement method executed by the second measurement device 100b. The biological information measurement method shown in FIG. 10 is appropriately executed at predetermined time intervals.

In step S201, the control unit 30 acquires motion data. The determination unit 37 of the control unit 30 acquires the motion data output from the motion sensor 50. The motion sensor 50 outputs motion data to the control unit 30 at a predetermined timing or when a predetermined amount of body motion is detected.

After acquiring the motion data, the control unit 30 determines whether the user M is in a sleeping state in step S203. The determination unit 37 determines whether the user M is in a sleeping state based on the acquired motion data. The determination unit 37 determines whether the user M is in the sleeping state, and generates a determination signal based on a determination result. When it is determined that the user M is in the sleeping state, the determination unit 37 generates a sleeping state signal as the determination signal. When it is determined that the user M is not in the sleeping state, the determination unit 37 may generate an active state signal as the determination signal. The determination unit 37 may generate a sleep displacement signal and an activity displacement signal based on the determination result. The determination unit 37 transmits the determination signal to the detection control unit 31 or the data processing unit 33. The determination unit 37 may transmit the determination signal to the detection control unit 31 and the data processing unit 33. When it is determined that the user M is not in the sleeping state, the control unit 30 proceeds to step S205 (step S203: NO). When it is determined that the user M is in the sleeping state, the control unit 30 proceeds to step S207 (step S203: YES).

In step S205, the control unit 30 measures the pulse wave in the first measurement mode. The detection control unit 31 of the control unit 30 operates the light emitting element unit 10 and the light receiving element unit 20 in the first measurement mode. The detection control unit 31 causes the green light emitting element 11c to emit light via the drive circuit 13. The green light emitting element 11c emits the green light GL to the measurement site of the user M. The light receiving element 21 of the light receiving element unit 20 receives the green light GL. The output circuit 23 generates a green light detection signal based on the green light GL. The output circuit 23 transmits the green light detection signal to the data processing unit 33. The data processing unit 33 acquires the green light detection signal. The data processing unit 33 measures the pulse wave using the green light detection signal.

When the pulse wave is measured in the first measurement mode, the control unit 30 may cause the red light emitting element 11a and the infrared light emitting element 11b to emit light or turn off the red light emitting element 11a and the infrared light emitting element 11b. The control unit 30 preferably turns off the red light emitting element 11a and the infrared light emitting element 11b. Power consumption of the second measurement device 100b is reduced.

In step S207, the control unit 30 measures the pulse wave in the second measurement mode. The detection control unit 31 of the control unit 30 operates the light emitting element unit 10 and the light receiving element unit 20 in the second measurement mode. The detection control unit 31 causes the red light emitting element 11a and the infrared light emitting element 11b to emit light via the drive circuit 13. The red light emitting element 11a emits the red light RL to the measurement site of the user M. The infrared light emitting element 11b emits the infrared light NL to the measurement site of the user M. The light receiving element 21 of the light receiving element unit 20 receives the red light RL and the infrared light NL. The output circuit 23 generates a red light detection signal based on the red light RL. The output circuit 23 generates an infrared light detection signal based on the infrared light NL. The output circuit 23 outputs the red light detection signal and the infrared light detection signal to the data processing unit 33. The data processing unit 33 acquires the red light detection signal and the infrared light detection signal. The data processing unit 33 measures the pulse wave using the red light detection signal or the infrared light detection signal. The data processing unit 33 measures the oxygen saturation concentration using the red light detection signal and the infrared light detection signal.

When the pulse wave is measured in the second measurement mode, the control unit 30 may cause the green light emitting element 11c to emit light or turn off the green light emitting element 11c. The control unit 30 preferably turns off the green light emitting element 11c. The power consumption of the second measurement device 100b is reduced by turning off the green light emitting element 11c. When the green light emitting element 11c emits light, the output circuit 23 preferably does not generate a green light detection signal. The data processing unit 33 does not execute data processing of measuring the pulse wave using the green light detection signal. The second measurement device 100b can prevent an increase in power consumption due to data processing.

The determination unit 37 may determine whether the user M is in the sleeping state based on the motion data and the quality data. As an example, the determination unit 37 acquires an output data amount, an accumulated data amount, and the like per unit time from the motion sensor 50 as the motion data. The determination unit 37 determines whether the user M is in a sleeping state by comparing a preset body motion amount threshold value with the motion data. When the motion data is equal to or larger than the body motion amount threshold value, the determination unit 37 determines that the user M is not in the sleeping state. The determination unit 37 determines that the user M is in the active state. When the motion data is smaller than the body motion amount threshold value, the determination unit 37 compares the quality data with the quality data threshold value. As an example, the determination unit 37 compares the correlation coefficient with the correlation coefficient threshold value. The determination unit 37 determines whether the user M is in a sleeping state by comparing the correlation coefficient with the correlation coefficient threshold value. When the correlation coefficient is smaller than the correlation coefficient threshold value, the determination unit 37 determines that the user M is not in the sleeping state. When the correlation coefficient is equal to or larger than the correlation coefficient threshold value, the determination unit 37 determines that the user M is in the sleeping state.

The determination unit 37 generates a determination signal such as a sleeping state signal indicating that the user M is in the sleeping state based on the motion data and the quality data. The determination unit 37 may generate a sleep displacement signal indicating displacement from an active state to a sleeping state as the determination signal based on a determination result. The determination unit 37 transmits the sleeping state signal or the sleep displacement signal to either the detection control unit 31 or the data processing unit 33. The determination unit 37 may transmit the sleeping state signal or the sleep displacement signal to the detection control unit 31 and the data processing unit 33. The determination unit 37 may generate an active state signal indicating that the user M is in an active state as the determination signal. The determination unit 37 may generate an activity displacement signal indicating displacement from a sleeping state to an active state as the determination signal. The determination unit 37 transmits the active state signal or the activity displacement signal to either the detection control unit 31 or the data processing unit 33. The determination unit 37 may transmit the active state signal or the activity displacement signal to the detection control unit 31 and the data processing unit 33.

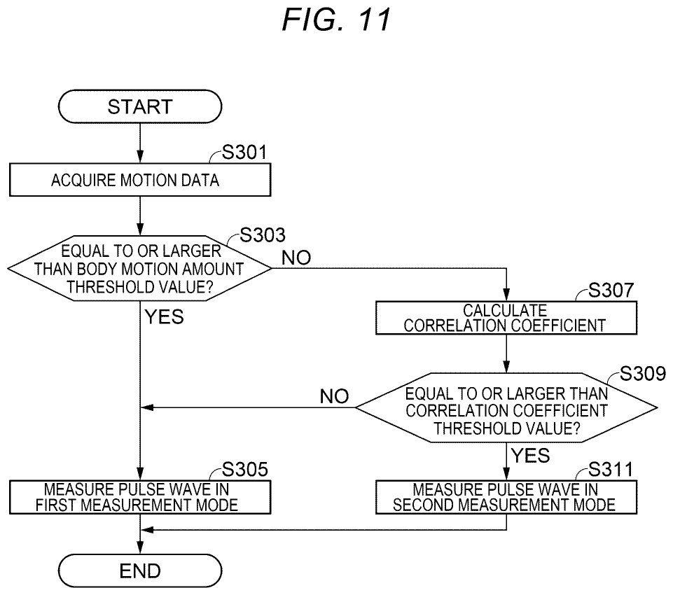

FIG. 11 shows a flowchart for measuring a pulse wave. The flowchart shown in FIG. 11 is an example of the biological information measurement method. FIG. 11 shows the biological information measurement method executed by the second measurement device 100b. The biological information measurement method shown in FIG. 11 is appropriately executed at predetermined time intervals.

In step S301, the control unit 30 acquires motion data. The determination unit 37 of the control unit 30 acquires the motion data output from the motion sensor 50. The motion sensor 50 outputs motion data to the control unit 30 at a predetermined timing or when a predetermined amount of body motion is detected.

After acquiring the motion data, the control unit 30 determines whether the motion data is equal to or larger than the body motion amount threshold value in step S303. The determination unit 37 compares the motion data with the body motion amount threshold value. The determination unit 37 reads the body motion amount threshold value from the memory 40. When it is determined that the motion data is equal to or larger than the body motion amount threshold value, the determination unit 37 determines that the user M is in an active state. The determination unit 37 transmits an active state signal or an activity displacement signal to the detection control unit 31. The control unit 30 proceeds to step S305 (step S303: YES). When it is determined that the motion data is smaller than the body motion amount threshold value, the determination unit 37 estimates that the user M is in a sleeping state. The control unit 30 proceeds to step S307 (step S303: NO).

In step S305, the control unit 30 measures the pulse wave in the first measurement mode. The detection control unit 31 of the control unit 30 receives the active state signal or the activity displacement signal from the determination unit 37. The detection control unit 31 operates the light emitting element unit 10 and the light receiving element unit 20 in the first measurement mode. The detection control unit 31 causes the green light emitting element 11c to emit light via the drive circuit 13. The green light emitting element 11c emits the green light GL to the measurement site of the user M. The light receiving element 21 of the light receiving element unit 20 receives the green light GL. The output circuit 23 generates a green light detection signal based on the green light GL. The output circuit 23 outputs the green light detection signal to the data processing unit 33. The data processing unit 33 acquires the green light detection signal. The data processing unit 33 measures the pulse wave using the green light detection signal.

When the pulse wave is measured in the first measurement mode, the control unit 30 may cause the red light emitting element 11a and the infrared light emitting element 11b to emit light or turn off the red light emitting element 11a and the infrared light emitting element 11b. The control unit 30 preferably turns off the red light emitting element 11a and the infrared light emitting element 11b. Power consumption of the second measurement device 100b is reduced.

In step S307, the control unit 30 calculates a correlation coefficient. The detection control unit 31 causes the red light emitting element lla and the infrared light emitting element 11b to emit light. The red light emitting element 1la emits the red light RL to the measurement site of the user M. The infrared light emitting element 11b emits the infrared light NL to the measurement site of the user M. The light receiving element 21 of the light receiving element unit 20 receives the red light RL and the infrared light NL. The output circuit 23 generates a red light detection signal based on the red light RL and an infrared light detection signal based on the infrared light NL. The output circuit 23 outputs the red light detection signal and the infrared light detection signal to the data processing unit 33. The data processing unit 33 of the control unit 30 acquires the red light detection signal and the infrared light detection signal. When the red light detection signal and the infrared light detection signal are acquired, the control unit 30 may or may not cause the green light emitting element 11c to emit light.

The control unit 30 acquires the red light detection signal and the infrared light detection signal. The data processing unit 33 calculates the correlation coefficient using the red light detection signal and the infrared light detection signal. In FIG. 11, the control unit 30 calculates the correlation coefficient, and may calculate sensitivity data instead of the correlation coefficient. The data processing unit 33 transmits the correlation coefficient to the determination unit 37. The determination unit 37 acquires the correlation coefficient.

After calculating the correlation coefficient, the control unit 30 determines whether the correlation coefficient is equal to or larger than the correlation coefficient threshold value in step S309. The determination unit 37 compares the correlation coefficient with the correlation coefficient threshold value. The determination unit 37 reads the correlation coefficient threshold value stored in the memory 40. When the correlation coefficient is smaller than the correlation coefficient threshold value, the determination unit 37 determines that the user M is in an active state. The determination unit 37 transmits an active state signal or an activity displacement signal to the detection control unit 31. The control unit 30 proceeds to step S305 and measures the pulse wave in the first measurement mode (step S309: NO). When the correlation coefficient is equal to or larger than the correlation coefficient threshold value, the determination unit 37 determines that the user M is in a sleeping state. The determination unit 37 determines that the user M is not in the active state. The determination unit 37 transmits a sleeping state signal or a sleep displacement signal to the detection control unit 31. The control unit 30 proceeds to step S311 (step S309: YES). In step S309 in FIG. 11, the correlation coefficient and the correlation coefficient threshold value are used to determine whether the user M is in the active state, but the present disclosure is not limited thereto. The sensitivity data and the sensitivity data threshold value may be used for determination.

In step S311, the control unit 30 measures the pulse wave in the second measurement mode. The detection control unit 31 of the control unit 30 operates the light emitting element unit 10 and the light receiving element unit 20 in the second measurement mode. The detection control unit 31 causes the red light emitting element 11a and the infrared light emitting element 11b to emit light via the drive circuit 13. The red light emitting element 11a emits the red light RL to the measurement site of the user M. The infrared light emitting element 11b emits the infrared light NL to the measurement site of the user M. The light receiving element 21 of the light receiving element unit 20 receives the red light RL and the infrared light NL. The output circuit 23 generates a red light detection signal based on the red light RL. The output circuit 23 generates an infrared light detection signal based on the infrared light NL. The output circuit 23 outputs the red light detection signal and the infrared light detection signal to the data processing unit 33. The data processing unit 33 acquires the red light detection signal and the infrared light detection signal. The data processing unit 33 measures the pulse wave using the red light detection signal or the infrared light detection signal. The data processing unit 33 measures the oxygen saturation concentration using the red light detection signal and the infrared light detection signal.

When the pulse wave is measured in the second measurement mode, the control unit 30 may cause the green light emitting element 11c to emit light or turn off the green light emitting element 11c. The control unit 30 preferably turns off the green light emitting element 11c. The power consumption of the second measurement device 100b is reduced by turning off the green light emitting element 11c. When the green light emitting element 11c emits light, the output circuit 23 preferably does not generate a green light detection signal. The data processing unit 33 does not execute data processing of measuring the pulse wave using the green light detection signal. The second measurement device 100b can prevent an increase in power consumption due to data processing.

### Third Embodiment

A third embodiment shows an example of the measurement system 1000 that analyzes an irregular pulse or the like in the tablet terminal 200. The measurement system 1000 according to the third embodiment includes the second measurement device 100b and the tablet terminal 200. The measurement system 1000 may include the first measurement device 100a instead of the second measurement device 100b.

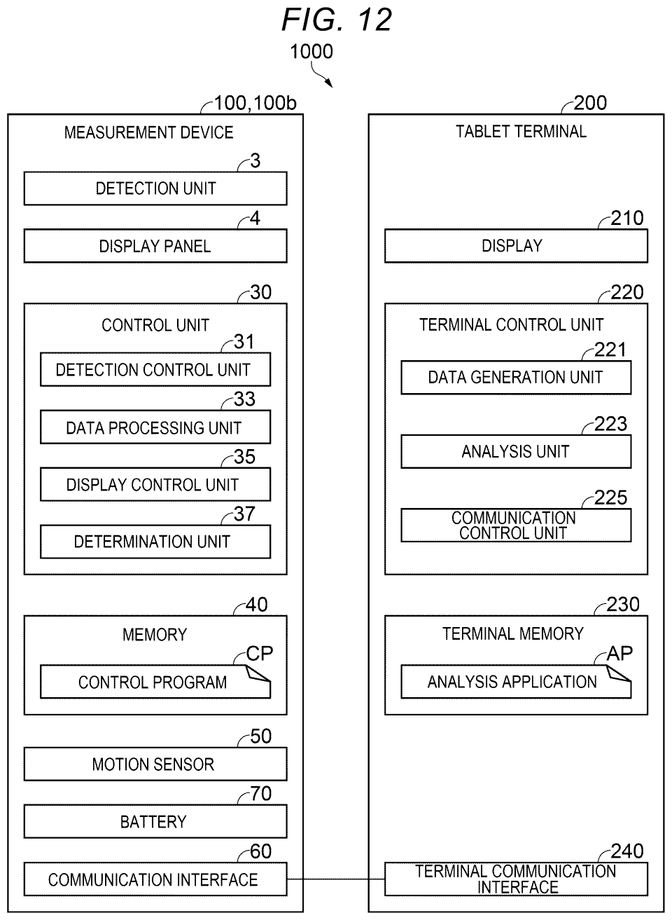

FIG. 12 shows a block configuration of the measurement system 1000. A configuration of the second measurement device 100b shown in FIG. 12 is the same as the configuration of the second measurement device 100b shown in

FIG. 9. The second measurement device 100b generates a detection signal under the control of the control unit 30. The control unit 30 operates the light emitting element unit 10 and the light receiving element unit 20 in the first measurement mode or in the second measurement mode. The control unit 30 generates a green light detection signal by operating the light emitting element unit 10 and the light receiving element unit 20 in the first measurement mode. The second measurement device 100b transmits the green light detection signal to the tablet terminal 200 via the communication interface 60. The communication interface 60 transmits a green light detection signal to the tablet terminal 200. The control unit 30 generates the red light detection signal and the infrared light detection signal by operating the light emitting element unit 10 and the light receiving element unit 20 in the second measurement mode. The second measurement device 100b transmits a red light detection signal or an infrared light detection signal to the tablet terminal 200 via the communication interface 60. The second measurement device 100b may transmit the red light detection signal and the infrared light detection signal to the tablet terminal 200 via the communication interface 60. The communication interface 60 transmits at least one of the red light detection signal and the infrared light detection signal to the tablet terminal 200.

The tablet terminal 200 can calculate the pulse wave and the oxygen saturation concentration. The tablet terminal 200 evaluates the irregular pulse based on the pulse wave. The tablet terminal 200 evaluates the sleep apnea syndrome based on the oxygen saturation concentration. The tablet terminal 200 includes the display 210, the terminal control unit 220, a terminal memory 230, and a terminal communication interface 240.

The terminal control unit 220 is a terminal control controller that controls operations of various units in the tablet terminal 200. The terminal control unit 220 is, for example, a terminal processor having a CPU. The terminal control unit 220 may include one or more processors. The terminal control unit 220 may include a semi-conductor memory such as a RAM or a ROM. The semi-conductor memory functions as a work area for the control unit 30. The terminal control unit 220 functions as a data generation unit 221, an analysis unit 223, and a communication control unit 225 by executing an analysis application AP stored in the terminal memory 230.

The data generation unit 221 is a functional unit that operates in the terminal control unit 220. The data generation unit 221 calculates biological information such as a pulse wave and an oxygen saturation concentration. When the data generation unit 221 acquires a green light detection signal from the second measurement device 100b, the data generation unit 221 calculates the pulse wave using the green light detection signal. When the data generation unit 221 acquires a red light detection signal or an infrared light detection signal from the second measurement device 100b, the data generation unit 221 calculates the pulse wave using the red light detection signal or the infrared light detection signal. When the data generation unit 221 acquires the red light detection signal and the infrared light detection signal from the second measurement device 100b, the data generation unit 221 calculates the oxygen saturation concentration using the red light detection signal and the infrared light detection signal. The data generation unit 221 calculates the oxygen saturation concentration by a similar method as the method executed by the data processing unit 33. The data generation unit 221 outputs the calculated pulse wave and oxygen saturation concentration to the analysis unit 223.

The analysis unit 223 is a functional unit that operates in the terminal control unit 220. The analysis unit 223 acquires the pulse wave output from the data generation unit 221. The analysis unit 223 evaluates an irregular pulse by analyzing the pulse wave. The analysis unit 223 outputs an evaluation result of the irregular pulse to the display 210. The analysis unit 223 may store the evaluation result of the irregular pulse in the terminal memory 230.

The analysis unit acquires biological 223 information such as an oxygen saturation concentration output from the data generation unit 221. As an example, the analysis unit 223 evaluates the sleep apnea syndrome by analyzing the oxygen saturation concentration. The analysis unit 223 outputs an evaluation result of the sleep apnea syndrome to the display 210. The analysis unit 223 may cause the terminal memory 230 to store the evaluation result of the sleep apnea syndrome. The analysis unit 223 may generate chart data using the oxygen saturation concentration. The analysis unit 223 outputs the generated chart data to the display 210. The display 210 displays various charts based on the chart data.

The communication control unit 225 is a functional unit that operates in the terminal control unit 220. The communication control unit 225 controls communication with the second measurement device 100b. The communication control unit 225 establishes communication connection with the second measurement device 100b. The communication control unit 225 causes the second measurement device 100b to transmit various types of data at a predetermined timing. The communication control unit 225 causes the second measurement device 100b to transmit the red light detection signal, the infrared light detection signal, and the green light detection signal at a predetermined timing.

The terminal memory 230 stores various types of data. The terminal memory 230 stores control data for operating various units in the tablet terminal 200. The terminal memory 230 may store various types of analysis data and the like analyzed by the terminal control unit 220. The terminal memory 230 stores the analysis application AP that operates in the terminal control unit 220. The terminal memory 230 stores the calibration table PT referred to by the data generation unit 221. The terminal memory 230 includes a ROM, a RAM and the like. The terminal memory 230 corresponds to an example of a terminal storage unit.

The analysis application AP operates various functional units by being executed by the terminal control unit 220. The analysis application AP causes the terminal control unit 220 to operate as the data generation unit 221, the analysis unit 223, and the communication control unit 225. The analysis application AP causes the terminal control unit 220 to operate as a functional unit other than the data generation unit 221, the analysis unit 223, and the communication control unit 225.

The terminal communication interface 240 is a terminal interface circuit for performing communication connection with the second measurement device 100b. The terminal communication interface 240 is connected to the second measurement device 100b in a wired or wireless manner according to a predetermined protocol. The terminal communication interface 240 includes, for example, a connection port for wired communication or an antenna for wireless communication. The terminal communication interface 240 receives various types of data such as the red light detection signal, the infrared light detection signal, and the green light detection signal from the second measurement device 100b. The terminal communication interface 240 transmits various types of control data for controlling an operation of the second measurement device 100b, information related to the user M, and the like to the second measurement device 100b. The terminal communication interface 240 may be communicably connected to an external device other than the second measurement device 100b. The terminal communication interface 240 corresponds to an example of a terminal communication circuit.

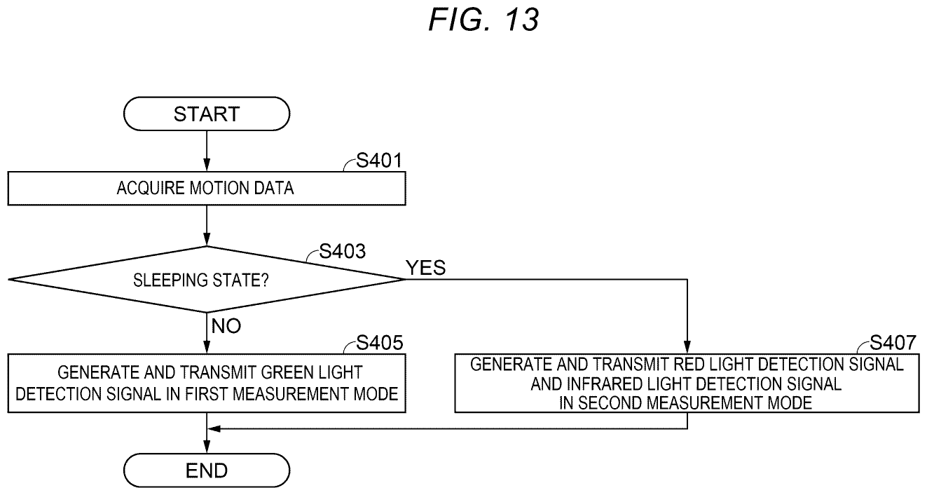

FIG. 13 shows an operation of the measurement device 100 in the measurement system 1000. FIG. 13 is a flowchart showing an operation of the second measurement device 100b according to the third embodiment. The flowchart shown in FIG. 13 shows a part of the biological information measurement method executed by the measurement system 1000.

In step S401, the second measurement device 100b acquires motion data. The determination unit 37 of the control unit 30 acquires the motion data output from the motion sensor 50. The motion sensor 50 outputs motion data to the control unit 30 at a predetermined timing or when a predetermined amount of body motion is detected.

After acquiring the motion data, the second measurement device 100b determines whether the user M is in a sleeping state in step S403. The determination unit 37 compares the motion data with the body motion amount threshold value. The determination unit 37 determines whether the user M is in a sleeping state by comparing the motion data with the body motion amount threshold value. The determination unit 37 determines whether the user M is in the sleeping state, and generates a determination signal based on a determination result. When it is determined that the user M is in the sleeping state, the determination unit 37 generates a sleeping state signal or a sleep displacement signal as a determination signal. When it is determined that the user M is not in the sleeping state, the determination unit 37 generates an active state signal or an activity displacement signal as the determination signal. The determination unit 37 transmits the determination signal to the detection control unit 31 and the data processing unit 33. The second measurement device 100b determines that the user M is not in the sleeping state when it is determined that the motion data is equal to or larger than the body motion amount threshold value. The second measurement device 100b proceeds to step S405 (step S403: NO). When it is determined that the motion data is smaller than the body motion amount threshold value, the second measurement device 100b determines that the user M is in the sleeping state. The second measurement device 100b proceeds to step S407 (step S403: YES).

In step S405, the second measurement device 100b generates and transmits a green light detection signal in the first measurement mode. The detection control unit 31 of the control unit 30 receives the active state signal or the activity displacement signal from the determination unit 37. The detection control unit 31 operates the light emitting element unit 10 and the light receiving element unit 20 in the first measurement mode. The detection control unit 31 causes the green light emitting element 11c to emit light via the drive circuit 13. The green light emitting element 11c emits the green light GL to the measurement site of the user M. The light receiving element 21 of the light receiving element unit 20 receives the green light GL. The output circuit 23 generates a green light detection signal based on the green light GL. The output circuit 23 outputs the green light detection signal to the data processing unit 33. The data processing unit 33 acquires the green light detection signal. The data processing unit 33 transmits the green light detection signal to the tablet terminal 200 via the communication interface 60.

When the green light detection signal in the first measurement mode is generated, the second measurement device 100b may cause the red light emitting element 11a and the infrared light emitting element 11b to emit light or turn off the red light emitting element 11a and the infrared light emitting element 11b. The second measurement device 100b preferably turns off the red light emitting element 11a and the infrared light emitting element 11b. Power consumption of the second measurement device 100b is reduced.

In step S407, the second measurement device 100b generates and transmits the red light detection signal and the infrared light detection signal in the second measurement mode. The detection control unit 31 of the control unit 30 operates the light emitting element unit 10 and the light receiving element unit 20 in the second measurement mode. The detection control unit 31 causes the red light emitting element 11a and the infrared light emitting element 11b to emit light via the drive circuit 13. The red light emitting element 11a emits the red light RL to the measurement site of the user M. The infrared light emitting element 11b emits the infrared light NL to the measurement site of the user M. The light receiving element 21 of the light receiving element unit 20 receives the red light RL and the infrared light NL. The output circuit 23 generates a red light detection signal based on the red light RL. The output circuit 23 generates an infrared light detection signal based on the infrared light NL. The output circuit 23 transmits the red light detection signal and the infrared light detection signal to the data processing unit 33. The data processing unit 33 acquires the red light detection signal and the infrared light detection signal. The data processing unit 33 transmits the red light detection signal and the infrared light detection signal to the tablet terminal 200 via the communication interface 60.

When the red light detection signal and the infrared light detection signal in the second measurement mode are generated, the second measurement device 100b may cause the green light emitting element 11c to emit light or turn off the green light emitting element 11c. The second measurement device 100b preferably turns off the green light emitting element 11c. The power consumption of the second measurement device 100b is reduced by turning off the green light emitting element 11c. When the green light emitting element 11c emits light, the output circuit 23 preferably does not generate a green light detection signal. The data processing unit 33 does not execute processing of calculating the pulse wave using the green light detection signal. The second measurement device 100b can prevent an increase in power consumption due to data processing.

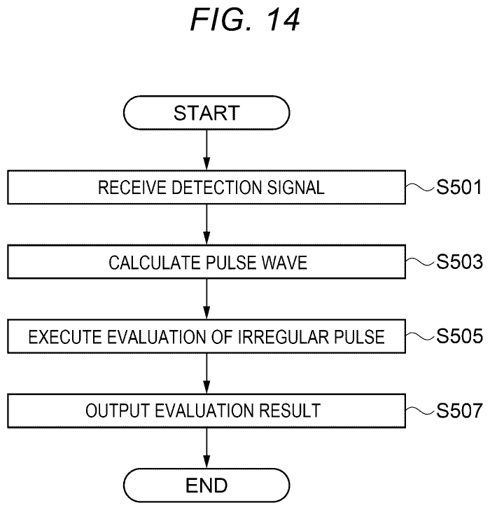

FIG. 14 shows an operation of the tablet terminal 200 in the measurement system 1000. FIG. 14 is a flowchart showing an operation of the tablet terminal 200 according to the third embodiment. The flowchart shown in FIG. 14 shows a part of the biological information measurement method executed by the measurement system 1000.

The tablet terminal 200 receives a detection signal in step S501. The terminal communication interface 240 of the tablet terminal 200 receives the detection signal transmitted via the communication interface 60. The terminal communication interface 240 receives a red light detection signal, an infrared light detection signal, and a green light detection signal as detection signals. When the second measurement device 100b generates the green light detection signal : in the first measurement mode, the terminal communication interface 240 receives the green light detection signal. When the second measurement device 100b generates the red light detection signal and the infrared light detection signal second measurement mode, the terminal communication interface 240 receives the red light detection signal and the infrared light detection signal. The terminal communication interface 240 may receive the red light detection signal, the infrared light detection signal, and the green light detection signal at a predetermined timing. The terminal communication interface 240 transmits the red light detection signal, the infrared light detection signal, and the green light detection signal to the data generation unit 221.

After receiving the detection signal, the tablet terminal 200 calculates a pulse wave in step S503. The data generation unit 221 acquires the red light detection signal, the infrared light detection signal, and the green light detection signal via the terminal communication interface 240. Upon receiving the green light detection signal, the data generation unit 221 calculates the pulse wave using the green light detection signal. Upon receiving the red light detection signal and the infrared light detection signal, the data generation unit 221 calculates the pulse wave using the red light detection signal or the infrared light detection signal. Upon receiving the red light detection signal and the infrared light detection signal, the data generation unit 221 calculates the oxygen saturation concentration using the red light detection signal and the infrared light detection signal. The oxygen saturation concentration is calculated by the data generation unit 221 in the same manner as the data processing unit 33 according to the first embodiment. The data generation unit 221 transmits the generated pulse wave and oxygen saturation concentration to the analysis unit 223.

After calculating the pulse wave, the tablet terminal 200 evaluates an irregular pulse in step S505. The analysis unit 223 analyzes the pulse wave. The analysis unit 223 evaluates an irregular pulse by analyzing the pulse wave. The analysis unit 223 transmits an evaluation result of the irregular pulse as an analysis result to the display 210 or the terminal memory 230.

In step S505, the tablet terminal 200 analyzes biological information such as the oxygen saturation concentration. As an example, the analysis unit 223 evaluates the sleep apnea syndrome using the oxygen saturation concentration. The analysis unit 223 transmits an evaluation result of the sleep apnea syndrome to the display 210 or the terminal memory 230.

The tablet terminal 200 executes an evaluation of the irregular pulse, and then outputs an analysis result in step S507. The display 210 displays the evaluation result of the irregular pulse as an output of the analysis result. The tablet terminal 200 may output the evaluation result of the irregular pulse by sound or the like. The tablet terminal 200 may display a chart or the like indicating the analysis result and a change in the pulse wave on the display 210.

The tablet terminal 200 may output the analysis result of the biological information in step S507. As an example, the tablet terminal 200 displays the evaluation result of the sleep apnea syndrome on the display 210. The tablet terminal 200 outputs evaluation results of various types of biological information calculated using the red light detection signal, the infrared light detection signal, and the green light detection signal.

The measurement system 1000 includes the second measurement device 100b and the tablet terminal 200. The second measurement device 100b includes: the light emitting element unit 10 including the red light emitting element 11a configured to emit the red light RL, the infrared light emitting element 11b configured to emit the infrared light NL, and the green light emitting element 11c configured to emit the green light GL; the light receiving element unit 20 configured to receive the red light RL, the infrared light NL, and the green light GL emitted by the light emitting element unit 10, and to generate a red light detection signal based on the red light RL, an infrared light detection signal based on the infrared light NL, and a green light detection signal based on the green light GL; the control unit 30 configured to operate the light emitting element unit 10 and the light receiving element unit 20; and the communication interface 60 configured to transmit the red light detection signal, the infrared light detection signal, and the green light detection signal. The tablet terminal 200 includes the terminal communication interface 240 that receives the red light detection signal, the infrared light detection signal, and the green light detection signal, and the terminal control unit 220 that calculates a pulse wave using any one of the red light detection signal, the infrared light detection signal, and the green light detection signal, and that evaluates an irregular pulse. The control unit 30 operates the light emitting element unit 10 and the light receiving element unit 20 in the first measurement mode of generating the green light detection signal or in the second measurement mode of generating the red light detection signal and the infrared light detection signal.

When the second measurement device 100b operates in the second measurement mode, the measurement system 1000 can omit data processing of calculating the pulse wave using the green light detection signal. Since the data processing of calculating the pulse wave using the green light detection signal is omitted, power consumption related to the data processing is reduced.

The terminal control unit 220 calculates the oxygen saturation concentration using the red light detection signal and the infrared light detection signal, and evaluates the sleep apnea syndrome.

The measurement system 1000 can calculate the oxygen saturation concentration when the user M is in a sleeping state. The measurement system 1000 can evaluate the sleep apnea syndrome.

What is claimed is:

1. A biological information measurement device comprising:
a light emitting unit including a first light emitting element configured to emit red light, a second light emitting element configured to emit infrared light, and a third light emitting element configured to emit green light;
a light receiving unit configured to receive the red light, the infrared light, and the green light emitted by the light emitting unit, and to generate a first light receiving signal based on the red light, a second light receiving signal based on the infrared light, and a third light receiving signal based on the green light; and
a controller configured to measure a pulse wave and an oxygen saturation concentration based on the first light receiving signal, the second light receiving signal, and the third light receiving signal, wherein
the controller operates the light emitting unit and the light receiving unit in a first mode of measuring the pulse wave using the third light receiving signal or in a second mode of measuring the pulse wave using the first light receiving signal or the second light receiving signal.

2. The biological information measurement device according to claim 1, wherein
the controller
calculates signal quality data using the first light receiving signal and the second light receiving signal,
operates the light emitting unit and the light receiving unit in the second mode when the signal quality data is equal to or larger than a predetermined threshold value, and
operates the light emitting unit and the light receiving unit in the first mode when the signal quality data is smaller than the threshold value.

3. The biological information measurement device according to claim 2, wherein
the signal quality data is correlation data between the first light receiving signal and the second light receiving signal, and
the threshold value is a correlation data threshold value.

4. The biological information measurement device according to claim 1, wherein
the controller turns off the first light emitting element and the second light emitting element when operating the light emitting unit and the light receiving unit in the first mode.

5. The biological information measurement device according to claim 1, wherein
the controller turns off the third light emitting element when operating the light emitting unit and the light receiving unit in the second mode.

6. A biological information measurement method comprising:
causing a first light emitting element configured to emit red light and a second light emitting element configured to emit infrared light to emit light;
generating a first light receiving signal based on the red light and a second light receiving signal based on the infrared light;
operating in a second mode of measuring a pulse wave using the first light receiving signal or the second light receiving signal; and
operating by switching to a first mode of measuring the pulse wave using a third light receiving signal based on green light emitted by a third light emitting element when a switching signal is received.

7. The biological information measurement method according to claim 6, further comprising:
measuring an oxygen saturation concentration using the first light receiving signal and the second light receiving signal when operating in the second mode.

8. A biological information measurement system comprising a biological information measurement device and a control device, wherein
the biological information measurement device includes
a light emitting unit including a first light emitting element configured to emit red light, a second light emitting element configured to emit infrared light, and a third light emitting element configured to emit green light,
a light receiving unit configured to receive the red light, the infrared light, and the green light emitted by the light emitting unit, and to generate a first light receiving signal based on the red light, a second light receiving signal based on the infrared light, and a third light receiving signal based on the green light,
a controller configured to operate the light emitting unit and the light receiving unit, and
a communication unit configured to transmit the first light receiving signal, the second light receiving signal, and the third light receiving signal,
the control device includes
a terminal communication circuit configured to receive the first light receiving signal, the second light receiving signal, and the third light receiving signal, and
a data controller configured to calculate a pulse wave using any one of the first light receiving signal, the second light receiving signal, and the third light receiving signal, and to evaluate an irregular pulse, and
the controller operates the light emitting unit and the light receiving unit in a first mode of generating the third light receiving signal or in a second mode of generating the first light receiving signal and the second light receiving signal.

9. The biological information measurement system according to claim 8, wherein
the data controller calculates an oxygen saturation concentration using the first light receiving signal and the second light receiving signal, and evaluates a sleep apnea syndrome.
